(19) United States
(12) Patent Application Publication
(10) Pub. No.: US 2026/0069653 A1
(43) Pub. Date: Mar. 12, 2026

(54) COMPLEMENT INHIBITOR DOSING REGIMENS

(71) Applicant: Apellis Pharmaceuticals, Inc., Waltham, MA (US)

(72) Inventors: Cedric Francois, Prospect, KY (US); Pascal Deschatelets, West Newton, MA (US); Federico Grossi, Needham, MA (US)

(21) Appl. No.: 18/707,159

(22) PCT Filed: Nov. 3, 2022
(86) PCT No.: PCT/US22/48890

§ 371 Date: May 2, 2024

Related U.S. Application Data
(60) Provisional application No. 63/275,274, filed on Nov. 3, 2021.

(51) Int. Cl. A61K 38/12; A61K 9/00; A61K 47/60; A61P 7/00

(52) U.S. Cl. CPC A61K 38/12; A61K 9/0019; A61K 47/60; A61P 7/00

(57) ABSTRACT

Complement is an arm of the innate immune system that plays an important role in defending the body against infectious agents. Although compositions that inhibit complement are known, there remains a need for compositions that can acutely inhibit complement. Methods and compositions comprising PEGylated compstatin analog for inhibiting complement are described.

### CROSS REFERENCE TO RELATED APPLICATIONS

The present application claims priority to U.S. Provisional Patent Application No. 63/275,274 filed Nov. 3, 2021, the entire contents of which are hereby incorporated by reference in their entirety.

### BACKGROUND

Complement is an arm of the innate immune system that plays an important role in defending the body against infectious agents. The complement system comprises more than 30 serum and cellular proteins that are involved in three major pathways, known as the classical, alternative, and lectin pathways. Although compositions that inhibit complement are known, there remains a need for compositions that can acutely inhibit complement.

### SUMMARY

In one aspect, the disclosure features a method of method of inhibiting complement in a subject. In some embodiments, the method of inhibiting complement in a subject comprises intravenously administering to a subject in need thereof about 100 mg to about 2500 mg of a PEGylated compstatin analog comprising a PEG of about 40 kD. In some embodiments, complement is inhibited or reduced (e.g., to a level that is about 100%, 90%, 80%, 70%, 60%, 50%, 40%, or lower, relative to a control level) for about 2 hours to about 336 hours after administration.

In another aspect, the disclosure features a method of treating a subject in need of treatment of a complement-mediated disorder, comprising intravenously administering to a subject in need thereof about 100 mg to about 2500 mg of a PEGylated compstatin analog comprising a PEG of about 40 kD, thereby treating the complement-mediated disorder.

In some embodiments, a method comprises intravenously administering a single dose of a PEGylated compstatin analog. In some embodiments, a method comprises administering two or more doses of a PEGylated compstatin analog.

In some embodiments, a single dose is an infusion.

In some embodiments, a method comprises intravenously administering about 200 mg, about 600 mg, about 1500 mg, or about 2300 mg of a PEGylated compstatin analog. In some embodiments, a method comprises intravenously administering about 200 mg, about 600 mg, about 1500 mg, or about 2300 mg of a PEGylated compstatin analog over about 30 minutes.

In some embodiments, a method comprises administering an infusion at a rate of about 6.5 mg/min to about 80 mg/min. In some embodiments, a method comprises administering an infusion at a rate of about 6.5 mg/min, about 20 mg/min, about 50 mg/min, or about 75 mg/min.

In some embodiments, a method comprises administering an infusion over a period of about 15 minutes to about 1 hour.

In some embodiments, complement inhibition is assessed by measuring level of complement activity in a serum sample of a subject.

In some embodiments, a level of complement activity is measured using an alternative pathway assay, a classical pathway assay, or both.

In some embodiments, a subject has or is at risk of a complement-mediated disorder. In some embodiments, a complement-mediated disorder is hemolytic anemia, warm antibody autoimmune hemolytic anemia, cold agglutinin disease, C3 glomerulopathy, Paroxysmal Nocturnal Hemoglobinuria (PNH), myasthenia gravis, glomerulonephritis, Neuromyelitis Optica (NMO), Amyotrophic lateral sclerosis (ALS), polyneuropathy, nephropathy, or vasculitis.

In some embodiments, a method optionally further comprises subcutaneously administering a PEGylated compstatin analog to a subject subsequent to an intravenous administration step.

In some embodiments, following an administering step, complement in a subject is inhibited or reduced (e.g., to a level that is about 100%, 90%, 80%, 70%, 60%, 50%, 40%, or lower, relative to a control level) for about 2 hours to about 336 hours after administration.

In some embodiments, a subject is in need of treatment for an exacerbation of a disorder.

In another aspect, the disclosure features a method of treating acute hemolysis in a subject, e.g., in a subject suffering from a complement-mediated disorder, e.g., in a subject suffering from PNH, comprising administering intensive therapy to the subject, wherein the intensive therapy comprises (i) intravenously administering between about 540 and about 2160 mg of a PEGylated compstatin analog comprising a PEG of about 40 kD to the subject or (ii) subcutaneously administering between about 540 and about 2160 mg of a PEGylated compstatin analog comprising a PEG of about 40 kD to the subject each day for three consecutive days.

In some embodiments, intensive therapy comprises intravenously administering between about 540 mg and about 2160 mg of a PEGylated compstatin analog comprising a PEG of about 40 kD to the subject. In some embodiments, intensive therapy comprises intravenously administering about 1080 mg of a PEGylated compstatin analog comprising a PEG of about 40 kD to the subject. In some embodiments, intensive therapy comprises subcutaneously administering between about 540 mg and about 2160 mg of a PEGylated compstatin analog comprising a PEG of about 40 kD to the subject each day for three consecutive days. In some embodiments, intensive therapy comprises subcutaneously administering about 1080 mg of a PEGylated compstatin analog comprising a PEG of about 40 kD to the subject each day for three consecutive days.

In some embodiments, prior to the acute hemolysis, a subject is being treated with about 1080 mg of a PEGylated compstatin analog comprising a PEG of about 40 kD administered subcutaneously twice weekly and after the intensive therapy the subject receives treatment with about 1080 mg of a PEGylated compstatin analog comprising a PEG of about 40 kD administered subcutaneously every 3 days. In some embodiments, prior to the acute hemolysis, a subject is being treated with about 1080 mg of a PEGylated compstatin analog comprising a PEG of about 40 kD administered subcutaneously every 3 days, and after the intensive therapy the subject receives treatment with about 1080 mg of a PEGylated compstatin analog comprising a PEG of about 40 kD administered subcutaneously three times per week. In some embodiments, prior to the acute hemolysis, a subject is being treated with a C5 inhibitor, and after the intensive therapy the subject receives treatment with about 1080 mg of a PEGylated compstatin analog comprising a PEG of about 40 kD administered subcutaneously twice weekly, every 3 days or three times per week. In some embodiments, prior to the acute hemolysis, a subject is being treated with a C5 inhibitor, and after the intensive therapy the subject receives treatment with about 1080 mg of a PEGylated compstatin analog comprising a PEG of about 40 kD administered subcutaneously twice weekly. In some embodiments, prior to the acute hemolysis, a subject is not being treated with a complement inhibitor, and after the intensive therapy the subject receives treatment with about 1080 mg of a PEGylated compstatin analog comprising a PEG of about 40 kD administered subcutaneously twice weekly, every 3 days or three times per week. In some embodiments, prior to the acute hemolysis, a subject is not being treated with a complement inhibitor, and after the intensive therapy the subject receives treatment with about 1080 mg of a PEGylated compstatin analog comprising a PEG of about 40 kD administered subcutaneously twice weekly.

In some embodiments, a subject continues treatment with the C5 inhibitor for 4 weeks after receiving the intensive therapy and then discontinues treatment with the C5 inhibitor.

In some embodiments, prior to the intensive therapy, a subject had an LDH level at least 2×ULN.

In some embodiments, a method optionally further comprises determining that the subject is experiencing acute hemolysis by a method comprising detecting an LDH level of at least 2×ULN in a blood sample obtained from the subject prior to administration of the intensive therapy

In some embodiments, a method optionally further comprises measuring LDH in a blood sample obtained from the subject within 2 weeks after the intensive therapy.

In some embodiments, a PEGylated compstatin analog comprises a PEG having at least two compstatin analog moieties attached thereto. In some embodiments, a PEGylated compstatin analog comprises a linear PEG having a compstatin analog moiety attached to each end. In some embodiments, each compstatin analog moiety comprises a cyclic peptide that comprises an amino acid sequence of one of SEQ ID NOs: 3-36, 37, 69, 70, 71, and 72.

In some embodiments, a PEGylated compstatin analog comprises one or more PEG moieties attached to one or more compstatin analog moieties, wherein: each compstatin analog moiety comprises a cyclic peptide having an amino acid sequence as set forth in any of SEQ ID NOs:3-36, extended by one or more terminal amino acids at a N-terminus, C-terminus, or both, wherein one or more amino acids has a side chain comprising a primary or secondary amine and is separated from a cyclic peptide by a rigid or flexible spacer optionally comprising an oligo(ethylene glycol) moiety; and each PEG is covalently attached via a linking moiety to one or more compstatin analog moieties, and wherein a linking moiety comprises an unsaturated alkyl moiety, a moiety comprising a nonaromatic cyclic ring system, an aromatic moiety, an ether moiety, an amide moiety, an ester moiety, a carbonyl moiety, an imine moiety, a thioether moiety, and/or an amino acid residue.

In some embodiments, each compstatin analog moiety comprises a cyclic peptide extended by one or more amino acids at a N-terminus, C-terminus, or both, wherein one or more amino acids is separated from a cyclic portion of a peptide by a rigid or flexible spacer that comprises 8-amino-3,6-dioxaoctanoic acid (AEEAc) or 11-amino-3,6,9-trioxaundecanoic acid.

In some embodiments, a cyclic peptide comprises an amino acid sequence of SEQ ID NO:28, and wherein a spacer comprises AEEAc. In some embodiments, a PEGylated compstatin analog comprises a structure depicted in FIG. 1.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 shows the structure of an exemplary PEGylated compstatin analog.

FIG. 2 depicts mean serum level of the PEGylated compstatin analog of FIG. 1 having a PEG of about 40 kilodaltons (kD) for the cohorts. The PEGylated compstatin analog is referred to as PEG in the drawing.

FIG. 3 depicts mean AH50 (U/mL) levels for the PEGylated compstatin analog cohorts. The PEGylated compstatin analog is referred to as PEG in the drawing.

FIG. 4 depicts a diagram of an exemplary treatment regimen for intensive therapy of patients with acute hemolysis (AH) using the the PEGylated compstatin analog of FIG. 1, having a PEG of about 40 kilodaltons (kD) (pegcetacoplan). Patients received intravenous (IV) (top path) or intensive subcutaneous (SC) (bottom path) dosing of pegcetacoplan. LDH=lactate dehydrogenase; Q3D=every 3 days; TIW=3 times weekly; ULN=upper limit of normal.

FIG. 5 depicts percent change in lactate dehydrogenase (LDH) from day 1 of intensive therapy in patients with acute hemolysis. Patients received intensive subcutaneous (SC) (dashed lines) or intravenous (IV) (solid lines) dosing of the PEGylated compstatin analog of FIG. 1, having a PEG of about 40 kilodaltons (kD). The PEGylated compstatin analog is referred to as PEG in the drawing.

FIG. 6 depicts mean hemoglobin (Hb (g/L)) levels from day 1 of intensive therapy in patients with acute hemolysis. Patients received intensive dosing of the PEGylated compstatin analog of FIG. 1, having a PEG of about 40 kilodaltons (kD). The top data line (squares) represents those patients who did not receive a red blood cell (RBC) transfusion (N=9), while the bottom data line (circles) represents those patients who did receive at least one RBC transfusion (N=4). The number of patients from which measurements of Hb levels were collected for each time point are indicated by the numbers at bottom, just above the X-axis (top row=no transfusion (Tx); bottom row=transfusion (Tx)). Dashed horizontal lines represent the male (top) and female (bottom) lower limit of normal (LLN). W1=week 1, W2=week 2, W3=week 3, W4=week 4.

### DEFINITIONS

Antibody: As used herein, the term “antibody” refers to an immunoglobulin or a derivative thereof containing an an immunoglobulin domain capable of binding to an antigen. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments of the invention the antibody is a fragment such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16: 535-539. The antibody may be partially or completely humanized. An antibody may be polyclonal or monoclonal, though for purposes of the present invention monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Combination therapy: The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered followed by one or more additional agents. In the separate administration protocol, two or more agents may be administered a few minutes apart, or a few hours apart, a few days apart, or a few weeks apart. In some embodiments, two or more agents may be administered 1-2 weeks apart.

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Concurrent administration: As used herein, the term “concurrent administration” with respect to two or more agents, e.g., therapeutic agents, is administration performed using doses and time intervals such that the administered agents are present together within the body, e.g., at one or more sites of action in the body, over a time interval in non-negligible quantities. The time interval can be minutes (e.g., at least 1 minute, 1-30 minutes, 30-60 minutes), hours (e.g., at least 1 hour, 1-2 hours, 2-6 hours, 6-12 hours, 12-24 hours), days (e.g., at least 1 day, 1-2 days, 2-4 days, 4-7 days, etc.), weeks (e.g., at least 1, 2, or 3 weeks, etc.). Accordingly, the agents may, but need not be, administered together as part of a single composition. In addition, the agents may, but need not be, administered essentially simultaneously (e.g., within less than 5 minutes, or within less than 1 minute apart) or within a short time of one another (e.g., less than 1 hour, less than 30 minutes, less than 10 minutes, approximately 5 minutes apart). According to various embodiments of the disclosure, agents administered within such time intervals may be considered to be administered at substantially the same time. In certain embodiments of the disclosure, concurrently administered agents are present at effective concentrations within the body (e.g., in the blood and/or at a site of local complement activation) over the time interval. When administered concurrently, the effective concentration of each of the agents needed to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. The non-negligible concentration of an agent may be, for example, less than approximately 5% of the concentration that would be required to elicit a particular biological response, e.g., a desired biological response.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

Sequential administration: As used herein, the term “sequential administration” of two or more agents refers to administration of two or more agents to a subject such that the agents are not present together in the subject's body, or at a relevant site of activity in the body, at greater than non-negligible concentrations. Administration of the agents may, but need not, alternate. Each agent may be administered multiple times.

Subject: As used herein, the term “subject” or “test subject” refers to any organism to which a provided compound or composition is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Systemic: As used herein, the term “systemic” in reference to complement components, refers to complement proteins that are synthesized by liver hepatocytes and enter the bloodstream, or are synthesized by circulating macrophages or monocytes or other cells and secreted into the bloodstream.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent can be an agent that, when administered to a subject, can prevent an undesired side effect, such as an immune response to a viral vector described herein. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. In some embodiments, a therapeutically effective amount of a substance is an amount that is sufficient, when administered to a subject suffering from or susceptible to a disease, disorder, and/or condition, to treat, prevent, and/or delay the onset of an undesired side effect, e.g., an immune response to a viral vector described herein. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e, providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed a complement-mediated disorder or is at increased risk of developing such a disorder relative to a member of the general population. A composition of the disclosure can be administered prophylactically, i.e., before development of any symptom or manifestation of the condition. Typically in this case the subject will be at risk of developing the condition.

Nucleic acid: The term “nucleic acid” includes any nucleotides, analogs thereof, and polymers thereof. The term “polynucleotide” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotide linkages”). The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. In some embodiments, the prefix poly- refers to a nucleic acid containing 2 to about 10,000, 2 to about 50,000, or 2 to about 100,000 nucleotide monomer units. In some embodiments, the prefix oligo- refers to a nucleic acid containing 2 to about 200 nucleotide monomer units.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” One of ordinary skill in the art understands that a “viral vector”, as described herein, includes viral components in addition to a transgene described herein, e.g., capsid proteins.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

The disclosure provides methods and compositions to inhibit complement over a defined time period, during which the level of complement inhibition is reduced relatively quickly following initiation of administration of a PEGylated compstatin analog described herein.

### I. Complement System

Complement is a system consisting of numerous plasma and cell-bound proteins that plays a significant role in both innate and adaptive immunity. The proteins of the complement system act in a series of enzymatic cascades through a variety of protein interactions and cleavage events. To facilitate understanding of the disclosure, and without intending to limit the invention in any way, this section provides an overview of complement and its pathways of activation. Further details are found, e.g., in Kuby Immunology, 6^{th} ed., 2006; Paul, W. E., Fundamental Immunology, Lippincott Williams & Wilkins; 6^{th} ed., 2008; and Walport M J., Complement. First of two parts. N Engl J Med., 344(14):1058-66, 2001.

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils. It will be understood that the names “C2a” and “C2b” used initially were subsequently reversed in the scientific literature.

The alternative pathway is initiated by and amplified at, e.g., microbial surfaces and various complex polysaccharides. In this pathway, hydrolysis of C3 to C3(H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death. If the TCC does not insert into a membrane, it can circulate in the blood as soluble sC5b-9 (sC5b-9). Levels of sC5b-9 in the blood may serve as an indicator of complement activation.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (fH), and C4b-binding protein (C4 bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Deficiencies or defects in complement regulatory protein(s) are involved in the pathogenesis of a variety of complement-mediated disorders.

### II. Compstatin Analogs

Methods of the disclosure include administration of compstatin analogs. Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus as shown in Table 1 (SEQ ID NO: 8). The term “compstatin” is used herein consistently with such usage (i.e., to refer to SEQ ID NO: 8). Compstatin analogs that have higher complement inhibiting activity than compstatin have been developed. See, e.g., WO2004/026328 (PCT/US2003/029653), Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), WO/2009/046198 (PCT/US2008/078593); WO/2010/127336 (PCT/US2010/033345) and discussion below.

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

Table 1 provides a non-limiting list of compstatin analogs useful in the present disclosure. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 1 are amidated at the C-terminus. Bold text is used to indicate certain modifications. Activity relative to compstatin is based on published data and assays described therein (WO2004/026328, WO2007044668, Mallik, 2005; Katragadda, 2006). In certain embodiments, the peptides listed in Table 1 are cyclized via a disulfide bond between the two Cys residues when used in the therapeutic compositions and methods of the disclosure. Alternate means for cyclizing the peptides are also within the scope of the disclosure.

TABLE 1

| | | SEQ | Activity | |
|---|---|---|---|---|
| | | ID | over | |
| Peptide | Sequence | NO: | compstatin | |
| Compstatin | H-ICVVQDWGHHRCT-CONH₂ | 8 | * | |
| Ac-compstatin | Ac-ICVVQDWGHHRCT-CONH₂ | 9 | 3x | more |
| Ac-V4Y/H9A | Ac-ICVYQDWGAHRCT-CONH₂ | 10 | 14x | more |
| Ac-V4W/H9A -OH | Ac-ICVWQDWGAHRCT-COOH | 11 | 27x | more |
| Ac-V4W/H9A | Ac-ICVWQDWGAHRCT-CONH₂ | 12 | 45x | more |
| Ac-V4W/H9A/T13dT-OH | Ac-ICVWQDWGAHRCdT-COOH | 13 | 55x | more |
| Ac-V4(2-Nal)/H9A | Ac-ICV(2-Nal)QDWGAHRCT-CONH₂ | 14 | 99x | more |
| Ac V4(2-Nal)/H9A-OH | Ac-ICV[(2-Nal)QDWGAHRCT-COOH | 15 | 38x | more |
| Ac V4(1-Nal)/H9A-OH | Ac-ICV(1-Nal)QDWGAHRCT-COOH | 16 | 30x | more |
| Ac-V42Igl/H9A | Ac-ICV(2-Igl)QDWGAHRCT-CONH₂ | 17 | 39x | more |
| Ac-V42Igl/H9A-OH | Ac-ICV(2-Igl)QDWGAHRCT-COOH | 18 | 37x | more |
| Ac-V4Dht/H9A-OH | Ac-ICVDhtQDWGAHRCT-COOH | 19 | 5x | more |
| Ac-V4(Bpa)/H9A-OH | Ac-ICV(Bpa)QDWGAHRCT-COOH | 20 | 49x | more |
| Ac-V4(Bpa)/H9A | Ac-ICV(Bpa)QDWGAHRCT-CONH₂ | 21 | 86x | more |
| Ac-V4(Bta)/H9A-OH | Ac-ICV(Bta)QDWGAHRCT-COOH | 22 | 65x | more |
| Ac-V4(Bta)/H9A | Ac-ICV(Bta)QDWGAHRCT-CONH₂ | 23 | 64x | more |
| Ac-V4W/H9(2-Abu) | Ac-ICVWQDWG(2-Abu)HRCT-CONH₂ | 24 | 64x | more |
| +G/V4W/H9A+AN-OH | H-GICVWQDWGAHRCTAN-COOH | 25 | 38x | more |
| Ac-V4(5fW)/H9A | Ac-ICV(5fW)QDWGAHRCT-CONH₂ | 26 | 31x | more |
| Ac-V4(5-MeW)/H9A | Ac-ICV(5-methyl-W)QDWGAHRCT-CONH₂ | 27 | 67x | more |
| Ac-V4(1-MeW)/H9A | Ac-ICV(1-methyl-W)QDWGAHRCT-CONH₂ | 28 | 264x | more |
| Ac-V4W/W7(5fW)/H9A | Ac-ICVWQD(5fW)GAHRCT-CONH₂ | 29 | 121x | more |
| Ac-V4(5fWV)/W7(5fWV)/H9A | Ac-ICV(5fW)QD(5fW)GAHRCT- CONH₂ | 30 | NA | |
| Ac-V4(5-MeW)/W7(5fW)H9A | Ac-ICV(5-methyl-W)QD(5fW)GAHRCT- | 31 | NA | |
| | CONH₂ | | | |
| Ac-V4(1MeW)/W7(5fW)/H9A | Ac-ICV(1-methyl-W)QD(5fW)GAHRCT- | 32 | 264x | more |
| | CONH₂ | | | |
| +G/V4(6fW)/W7(6fW)H9A+N- | H-GICV(6fW)QD(6fW)GAHRCTN-COOH | 33 | 126x | more |
| OH | | | | |
| Ac-V4(1-formyl-W)/H9A | Ac-ICV(1-formyl-W)QDWGAHRCT-CONH₂ | 34 | 264x | more |
| Ac-V4(5-methoxy-W)/H9A | Ac-ICV(1-methyoxy-W)QDWGAHRCT- | 35 | 76x | more |
| | CONH₂ | | | |
| G/V4(5f-WV)/WV7(5fW)/ | H-GICV(5fW)QD(5fW)GAHRCTN-COOH | 36 | 112x | more |
| H9A+N-OH | | | | |

NA = not available

In certain embodiments of the compositions and methods of the disclosure, the compstatin analog has a sequence selected from sequences 9-36. In some embodiments, the compstatin analog has a sequence of SEQ ID NO: 28. As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

In some embodiments, one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin. In some embodiments, one or more of the compstatin analogs in Table 1 contains at least one N-methyl isoleucine, e.g., at the position corresponding to position 13 of compstatin. For example, a Thr at or near the C-terminal end of a peptide whose sequence is listed in Table 1 or any other compstatin analog sequence may be replaced by N-methyl Ile. As will be appreciated, in some embodiments the N-methylated amino acids comprise N-methyl Gly at position 8 and N-methyl Ile at position 13.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g., to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, CA, which also provides details of appropriate conjugation procedures.

In some embodiments, a compstatin analog of any of SEQ ID NOs: 9-36, is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, which facilitate conjugation with a reactive functional group to attach a PEG to the compstatin analog. In some embodiments, the compstatin analog comprises an amino acid having a side chain comprising a primary or secondary amine, e.g., a Lys residue. For example, a Lys residue, or a sequence comprising a Lys residue, is added at the N-terminus and/or C-terminus of a compstatin analog described herein (e.g., a compstatin analog comprising any one of SEQ ID NOs: 9-36).

In some embodiments, the Lys residue is separated from the cyclic portion of the compstatin analog by a rigid or flexible spacer. The spacer may, for example, comprise a substituted or unsubstituted, saturated or unsaturated alkyl chain, oligo(ethylene glycol) chain, and/or other moieties, e.g., as described herein with regard to linkers. The length of the chain may be, e.g., between 2 and 20 carbon atoms. In other embodiments the spacer is a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In some embodiments, the polymeric backbone or scaffold comprises multiple reactive functional groups, such as carboxylic acids, anhydride, or succinimide groups. The polymeric backbone or scaffold is reacted with the compstatin analogs. In some embodiments, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In some embodiments, short chains are prepolymerized, functionalized, and then a mixture of short chains of different composition are assembled into longer polymers.

In some embodiments, a compstatin analog moiety is attached at each end of a linear PEG. A bifunctional PEG having a reactive functional group at each end of the chain may be used, e.g., as described herein. In some embodiments, the reactive functional groups are identical while in some embodiments different reactive functional groups are present at each end.

In general and for compounds depicted herein, a polyethylene glycol moiety is drawn with the oxygen atom on the right side of the repeating unit or the left side of the repeating unit. In cases where only one orientation is drawn, the present disclosure encompasses both orientations (i.e., (CH₂CH₂O)ₙ and (OCH₂CH₂)ₙ) of polyethylene glycol moieties for a given compound or genus, or in cases where a compound or genus contains multiple polyethylene glycol moieties, all combinations of orientations are encompasses by the present disclosure.

In some embodiments a bifunctional linear PEG comprises a moiety comprising a reactive functional group at each of its ends. The reactive functional groups may be the same (homobifunctional) or different (heterobifunctional). In some embodiments the structure of a bifunctional PEG may be symmetric, wherein the same moiety is used to connect the reactive functional group to oxygen atoms at each end of the —(CH₂CH₂O)ₙ chain. In some embodiments different moieties are used to connect the two reactive functional groups to the PEG portion of the molecule. The structures of exemplary bifunctional PEGs are depicted below. For illustrative purposes, formulas in which the reactive functional group(s) comprise an NHS ester are depicted, but other reactive functional groups could be used.

In some embodiments, a bifunctional linear PEG is of formula A:

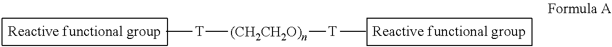

wherein each T and “Reactive functional group” is independently as defined below, and described in classes and subclasses herein, and n is as defined above and described in classes and subclasses herein.
Each T is independently a covalent bond or a C₁₋₁₂ straight or branched, hydrocarbon chain wherein one or more carbon units of T are optionally and independently replaced by —O—, —S—, —N(R^{x})—, —C(O)—, —C(O)O—, —OC(O)—, —N(R^{x})C(O)—, —C(O)N(R^{x})—, —S(O)—, —S(O)₂—, —N(R^{x})SO₂—, or —SO₂N(R^{x})—; and each R^{x} is independently hydrogen or C₁₋₆ aliphatic.
The Reactive functional group has the structure —COO—NHS.

Exemplary bifunctional PEGs of formula A include:

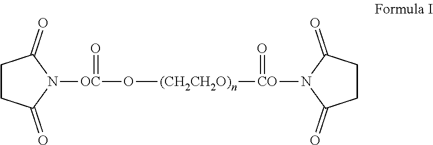

In some embodiments, a functional group (for example, an amine, hydroxyl, or thiol group) on a compstatin analog is reacted with a PEG-containing compound having a “reactive functional group” as described herein, to generate such conjugates. By way of example, Formula I can form compstatin analog conjugates having the structure:

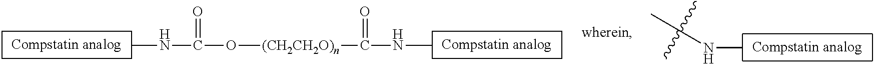

represents the attachment point of an amine group on a compstatin analog. In certain embodiments, an amine group is a lysine side chain group.

In certain embodiments, the PEG component of such conjugates has an average molecular weight of about 5 kD, about 10 kD, about 15 kD, about 20 kD, about 30 kD, or about 40 kD. In certain embodiments, the PEG component of such conjugates has an average molecular weight of about 40 kD.

The term “bifunctional” or “bifunctionalized” is sometimes used herein to refer to a compound comprising two compstatin analog moieties linked to a PEG. Such compounds may be designated with the letter “BF”. In some embodiments a bifunctionalized compound is symmetrical. In some embodiments the linkages between the PEG and each of the compstatin analog moieties of a bifunctionalized compound are the same. In some embodiments, each linkage between a PEG and a compstatin analog of a bifunctionalized compound comprises a carbamate. In some embodiments, each linkage between a PEG and a compstatin analog of a bifunctionalized compound comprises a carbamate and does not comprise an ester. In some embodiments, each compstatin analog of a bifunctionalized compound is directly linked to a PEG via a carbamate. In some embodiments, each compstatin analog of a bifunctionalized compound is directly linked to a PEG via a carbamate, and the bifunctionalized compound has the structure:

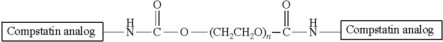

In some embodiments of formulae and embodiments described herein,

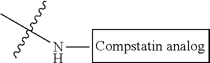

represents point of attachment of a lysine side chain group in a compstatin analog having the structure:

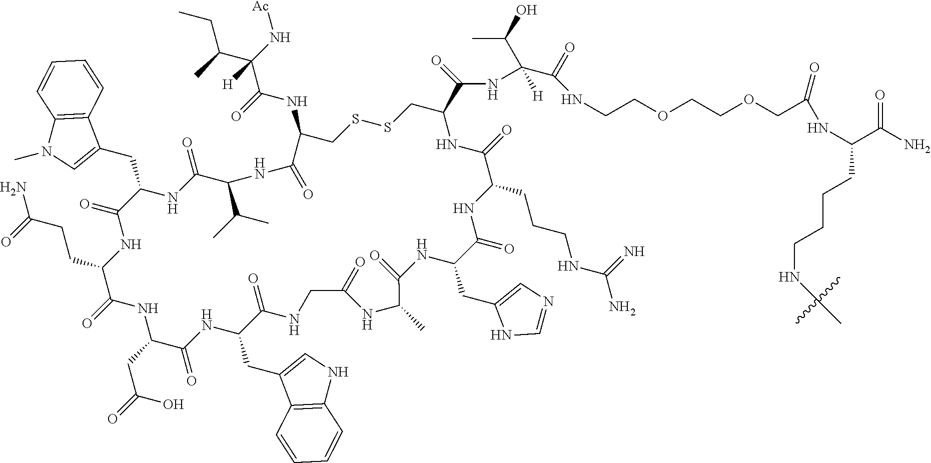 [SMILES: *NCCCC[C@H](NC(=O)COCCOCCNC(=O)[C@@H](NC(=O)[C@@H]1CSSC[C@H](NC(=O)[C@@H](NC(C)=O)[C@@H](C)CC)C(=O)N[C@@H](C(C)C)C(=O)N[C@@H](Cc2cn(C)c3ccccc23)C(=O)N[C@@H](CCC(N)=O)C(=O)N[C@@H](CC(=O)O)C(=O)N[C@@H](Cc2c[nH]c3ccccc23)C(=O)NCC(=O)N[C@@H](C)C(=O)N[C@@H](Cc2cnc[nH]2)C(=O)N[C@@H](CCCNC(=N)N)C(=O)N1)[C@@H](C)O)C(N)=O]

wherein the symbol “ [symbol] ” denotes the point of attachment of a chemical moiety to the remainder of a molecule or chemical formula.

PEGs comprising one or more reactive functional groups may, in some embodiments, be obtained from, e.g., NOF America Corp. White Plains, NY or BOC Sciences 45-16 Ramsey Road Shirley, NY 11967, USA, among others, or may be prepared using methods known in the art.

In some embodiments, a linker is used to connect a compstatin analog described herein and a PEG described herein. Suitable linkers for connecting a compstatin analog and a PEG are extensively described above and in classes and subclasses herein. In some embodiments, a linker has multiple functional groups, wherein one functional group is connected to a compstatin analog and another is connected to a PEG moiety. In some embodiments, a linker is a bifunctional compound. In some embodiments, a linker has the structure of NH₂(CH₂CH₂O)nCH₂C(═O)OH, wherein n is 1 to 1000. In some embodiments, a linker is 8-amino-3,6-dioxaoctanoic acid (AEEAc). In some embodiments, a linker is activated for conjugation with a polymer moiety or a functional group of a compstatin analog. For example, in some embodiments, the carboxyl group of AEEAc is activated before conjugation with the amine group of the side chain of a lysine group.

In some embodiments, a suitable functional group (for example, an amine, hydroxyl, thiol, or carboxylic acid group) on a compstatin analog is used for conjugation with a PEG moiety, either directly or via a linker. In some embodiments, a compstatin analog is conjugated through an amine group to a PEG moiety via a linker. In some embodiments, an amine group is the α-amino group of an amino acid residue. In some embodiments, an amine group is the amine group of the lysine side chain. In some embodiments, a compstatin analog is conjugated to a PEG moiety through the amino group of a lysine side chain (s-amino group) via a linker having the structure of NH₂(CH₂CH₂O)nCH₂C(═O)OH, wherein n is 1 to 1000. In some embodiments, a compstatin analog is conjugated to the PEG moiety through the amino group of a lysine side chain via an AEEAc linker. In some embodiments, the NH₂(CH₂CH₂O)nCH₂C(═O)OH linker introduces a —NH(CH₂CH₂O)nCH₂C(═O)— moiety on a compstatin lysine side chain after conjugation. In some embodiments, the AEEAc linker introduces a —NH(CH₂CH₂O)₂CH₂C(═O)— moiety on a compstatin lysine side chain after conjugation.

In some embodiments, a compstatin analog is conjugated to a PEG moiety via a linker, wherein the linker comprises an AEEAc moiety and an amino acid residue. In some embodiments, a compstatin analog is conjugated to a PEG moiety via a linker, wherein the linker comprises an AEEAc moiety and a lysine residue. In some embodiments, the C-terminus of a compstatin analog is connected to the amino group of AEEAc, and the C-terminus of AEEAc is connected to a lysine residue. In some embodiments, the C-terminus of a compstatin analog is connected to the amino group of AEEAc, and the C-terminus of AEEAc is connected to the α-amino group of a lysine residue. In some embodiments, the C-terminus of a compstatin analog is connected to the amino group of AEEAc, the C-terminus of AEEAc is connected to the α-amino group of the lysine residue, and a PEG moiety is conjugated through the F-amino group of said lysine residue. In some embodiments, the C-terminus of the lysine residue is modified. In some embodiments, the C-terminus of the lysine residue is modified by amidation. In some embodiments, the N-terminus of a compstatin analog is modified. In some embodiments, the N-terminus of a compstatin analog is acetylated.

In certain embodiments a compstatin analog may be represented as M-AEEAc-Lys-B₂, wherein B₂ is a blocking moiety, e.g., NH₂, M represents any of SEQ ID NOs: 9-36, with the proviso that the C-terminal amino acid of any of SEQ ID NOs: 9-36 is linked via a peptide bond to AEEAc-Lys-B₂. The NHS moiety of a monofunctional or multifunctional (e.g., bifunctional) PEG reacts with the free amine of the lysine side chain to generate a monofunctionalized (one compstatin analog moiety) or multifunctionalized (multiple compstatin analog moieties) PEGylated compstatin analog. In various embodiments any amino acid comprising a side chain that comprises a reactive functional group may be used instead of Lys (or in addition to Lys). A monofunctional or multifunctional PEG comprising a suitable reactive functional group may be reacted with such side chain in a manner analogous to the reaction of NHS-ester activated PEGs with Lys.

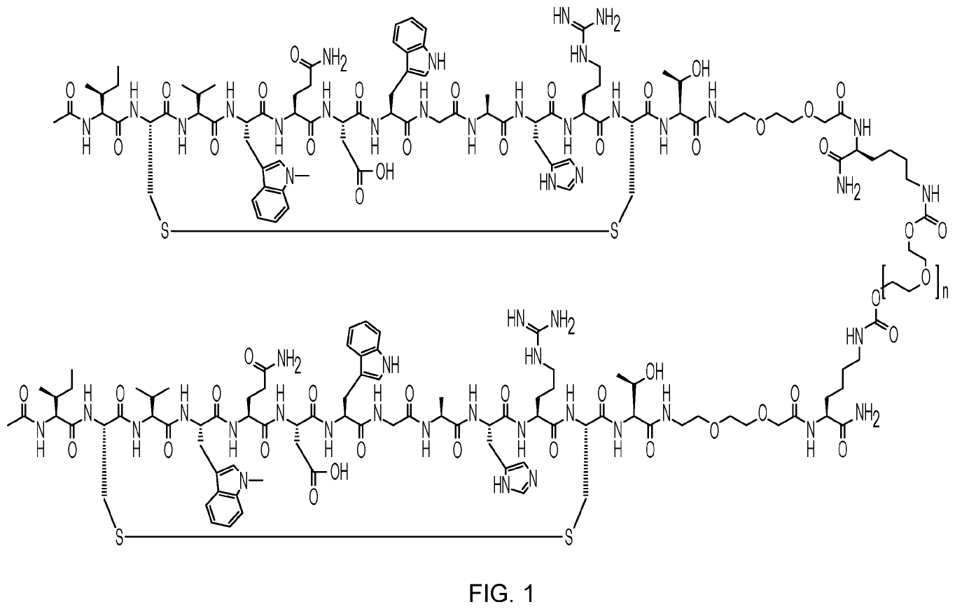

With regard to any of the above formulae and structures, it is to be understood that embodiments in which the compstatin analog component comprises any compstatin analog described herein, e.g., any compstatin analog of SEQ ID NOs; 9-36 are expressly disclosed. For example, and without limitation, a compstatin analog may comprise the amino acid sequence of SEQ ID NO: 28. An exemplary PEGylated compstatin analog in which the compstatin analog component comprises the amino acid sequence of SEQ ID NO: 28 is depicted in FIG. 1. It will be understood that the PEG moiety may have a variety of different molecular weights or average molecular weights in various embodiments, as described herein. In certain embodiments of particular interest, a compstatin analog is pegcetacoplan (“APL-2”), having the structure of the compound of FIG. 1 with n of about 800 to about 1100 and a PEG having an average molecular weight of about 40 kD. Pegcetacoplan is also referred to as Poly(oxy-1,2-ethanediyl), α-hydro-ω-hydroxy-, 15,15′-diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-N⁶-carboxy-L-lysinamide cyclic (2-->12)-(disulfide); or O,O′-bis[(S²,S¹²-cyclo{N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-L-lysinamide})-N^{6.15}-carbonyl]polyethylene glycol (n=800-1100). Additional compstatin analogs are described in, e.g., WO 2012/155107, WO 2014/078731, and WO 2019/166411.

### III. Administration Methods

In some embodiments, a compstatin analog described herein, e.g., a PEGylated compstatin analog described herein, e.g., pegcetacoplan, is intravenously administered to a subject. In some embodiments, a subject is in need of a relatively quick inhibition of complement (e.g., within about 15 mins, 30 mins, 45 mins, 1 hour, or 2 hours of administration) and/or complement inhibition over a defined period of time (e.g., for at least about 4 hours, 8 hours, 12 hours, 24 hours, 48 hours, 72 hours, 144 hours, 168 hours, or more following start of administration). In any of the embodiments described herein relating to administration of a PEGylated compstatin analog, the PEGylated compstatin analog may be pegcetacoplan

In some embodiments, a PEGylated compstatin analog, e.g., pegcetacoplan, is administered intravenously to a subject in need thereof at about 100 mg to about 2500 mg (e.g., about 100 mg to about 600 mg, about 600 mg to about 1500 mg, about 1500 mg to about 2500 mg, about 200 mg to about 2300 mg, about 100-120 mg, about 120-140 mg, about 140-160 mg, about 160-180 mg, about 180-200 mg, about 200-220 mg, about 220-240 mg, about 240-260 mg, about 260-280 mg, about 280-300 mg, about 300-320 mg, about 320-340 mg, about 340-360 mg, about 360-380 mg, about 380-400 mg, about 400-420 mg, about 420-440 mg, about 440-460 mg, about 460-480 mg, about 480-500 mg, about 500-520 mg, about 520-540 mg, about 540-560 mg, about 560-580 mg, about 580-600 mg, about 600-620 mg, about 620-640 mg, about 640-660 mg, about 660-680 mg, about 680-700 mg, about 700-720 mg, about 720-740 mg, about 740-760 mg, about 760-780 mg, about 780-800 mg, about 800-820 mg, about 820-840 mg, about 840-860 mg, about 860-880 mg, about 880-900 mg, about 900-920 mg, about 920-940 mg, about 940-960 mg, about 960-980 mg, about 980-1000 mg, about 1000-1020 mg, about 1020-1040 mg, about 1040-1060 mg, about 1060-1080 mg, about 1080-1100 mg, about 1100-1120 mg, about 1120-1140 mg, about 1140-1160 mg, about 1160-1180 mg, about 1180-1200 mg, about 1200-1250 mg, about 1250-1300 mg, about 1300-1350 mg, about 1350-1400 mg, about 1400-1450 mg, about 1450-1500 mg, about 1500-1550 mg, about 1550-1600 mg, about 1600-1650 mg, about 1650-1700 mg, about 1700-about 1750 mg, about 1750-1800 mg, about 1800-1850 mg, about 1850-1900 mg, about 1900-1950 mg, about 1950-2000 mg, about 2000-2050 mg, about 2050-2100 mg, about 2100-2150 mg, about 2150-2200 mg, about 2200-2250 mg, about 2250-2300 mg, about 2300-2350 mg, about 2350-2400 mg, about 2400-2450 mg, about 2450-2500 mg) or more. In some embodiments, a PEGylated compstatin analog, e.g., pegcetacoplan, is administered intravenously to a subject at about 200 mg. In some embodiments, a PEGylated compstatin analog, e.g., pegcetacoplan, is administered intravenously to a subject at about 600 mg. In some embodiments, a PEGylated compstatin analog is administered intravenously to a subject at about 1500 mg. In some embodiments, a PEGylated compstatin analog is administered intravenously to a subject at about 2300 mg. In some embodiments, complement is inhibited for at least about 4 hours, 8 hours, 12, hours, 16 hours, 20 hours, 24 hours, 28 hours, 32 hours, 36 hours, 48 hours, 72 hours, 144 hours, 168 hours, 192 hours, 216 hours, 240 hours, 264 hours, 288 hours, 312 hours, 336 hours, or more, following start of intravenous administration. In some embodiments, following intravenous administration of a PEGylated compstatin analog, complement is inhibited or reduced (e.g., to a level that is about 100%, 90%, 80%, 70%, 60%, 50%, 40%, or lower, relative to a control level). In some embodiments, following intravenous administration of a PEGylated compstatin analog, AH50 and/or CH50 is inhibited or reduced (e.g., to a level that is about 100%, 90%, 80%, 70%, 60%, 50%, 40%, or lower, relative to a control level). In some embodiments, following intravenous administration of a PEGylated compstatin analog, complement is not inhibited or reduced after about 168 hours, 192 hours, 216 hours, 240 hours, 264 hours, 288 hours, 312 hours, 336 hours, 360 hours, 408 hours, 456 hours, 504 hours, or more after administration. In some embodiments, a PEGylated compstatin analog is intravenously administered over a duration of time, e.g., as an infusion described herein, and complement is inhibited or reduced (e.g., to a level that is about 100%, 90%, 80%, 70%, 60%, 50%, 40%, or lower, relative to a control level) within about 30 minutes, 45 minutes, 1 hour, 2 hours, 3 hours, or 4 hours of the initiation of administration. In some embodiments, a control level is a level detected or measured in the same subject prior to intravenous administration of the PEGylated compstatin analog. In some embodiments, a control level is a reference level.

In some embodiments, a PEGylated compstatin analog is intravenously administered as a single dose. In some embodiments, the single dose is a bolus. In some embodiments, a bolus is an amount of a PEGylated compstatin analog that is administered, e.g., by IV infusion, over about 2 hours, 1 hour, 45 minutes, 30 minutes, 20 minutes, 10 minutes, 5 minutes, or less.

In some embodiments, a PEGylated compstatin analog is administered as an infusion at a rate of about 0.25 mg/min to about 85 mg/min, e.g., about 6.5 mg/min to about 80 mg/min, about 20 mg/min to about 50 mg/min. In some embodiments, the infusion rate is about 5 mg/min, about 6 mg/min, about 6.5 mg/min, about 7 mg/min, about 20 mg/min, about 50 mg/min, about 70 mg/min, about 75 mg/min, or about 80 mg/min. In some embodiments, the infusion is administered over a period of about 15 minutes to about 2 hours, e.g., about 15 minutes, about 30 minutes, about 45 minutes, about 1 hour, or about 2 hours.

In some embodiments, a PEGylated compstatin analog is intravenously administered as two or more doses. For example, any of the amounts described above may be administered as two doses administered intravenously close together in time, e.g., up to 30 minutes apart, up to 60 minutes apart, up to 2 hours apart, or up to 12 hours apart. For example, in some embodiments an amount of about 2160 mg may be administered as two doses, each of about 1080 mg, administered up to about 12 hours apart. In some embodiments, a first dose (e.g., a loading dose) and a second dose (e.g., a maintenance dose) are intravenously administered. In some embodiments, the first dose and the second dose comprise the same amount of the PEGylated compstatin analog. In some embodiments, the first dose and the second dose comprise different amounts of the PEGylated compstatin analog.

In some embodiments, the subject is an adult (i.e., the subject is at least 18 years old.) In some embodiments, the age of the subject is less than 18 years. In some embodiments, the age of the subject is at least 12 years. In some embodiments, the age of the subject is between 12-18 (e.g., between 12-17) years. In some embodiments, the age of the subject is between 6-12 years. In some embodiments, the age of the subject is between 1-12 years.

In some embodiments, a method of treating a complement-mediated disorder comprises administering multiple doses of a PEGylated compstatin analog described herein, e.g., pegcetacoplan, wherein a dose of between 500 mg and 2500 mg is administered intravenously, and additional doses are administered subcutaneously, with the first subcutaneous (SC) dose being administered within three days of the intravenous (IV) dose, e.g., within 3 days after administration of the IV dose. The first dose may serve as a loading dose, which rapidly (e.g., within 1 hour after administration) achieves a target serum concentration of the compstatin analog and rapidly reduces complement activation. Subsequent doses, which may be referred to maintenance therapy, may be administered to maintain therapeutically effective levels of the PEGylated compstatin analog, e.g., pegcetacoplan. In some embodiments a loading dose of pegcetacoplan is sufficient to result in a pegcetacoplan serum concentration of between about 500 μg/mL and about 800 μg/mL, e.g., between about 500 μg/mL and about 625 μg/mL, or between about 625 μg/mL and about 750 μg/mL, within 1 hour following administration. In some embodiments a complement-mediated disorder may be treated by administering an IV loading dose of a PEGylated compstatin analog, e.g., pegcetacoplan, followed by subcutaneously administered maintenance therapy. In certain embodiments a subject in need of treatment of a complement-mediated disorder receives an IV dose(s) of a PEGylated compstatin analog, e.g., pegcetacoplan, on a given day, which may be referred to as Day 1, then receives a first SC dose of pegcetacoplan on Day 2, Day 3, or Day 4, and thereafter continues treatment with SC administration of a PEGylated compstatin analog, e.g., pegcetacoplan, according to any of the SC dosing regimens described herein. For example, pegcetacoplan may be administered subcutaneously twice weekly, every three days, thrice weekly, or weekly at a dosage of between 990 mg and 1215 mg per dose, e.g., about 1080 mg per dose, as described in PCT/US2018/026753 (WO2018187813) and U.S. Pat. No. 11,040,107. In some embodiments the SC doses are administered in a volume of about 20 mL at a concentration of about 54 mg/mL. In some embodiments the SC doses (e.g., 1080 mg in a volume of 20 mL) are administered using a syringe pump or on-body delivery device. In some embodiments the patient may self-administer the SC doses.

In some embodiments a compstatin analog, e.g., a PEGylated compstatin analog, e.g., pegcetacoplan, is administered intravenously as described herein to a subject suffering from a complement-mediated disorder who is experiencing an exacerbation of the disease. An “exacerbation”, which may also be referred to as a “flare-up”, “attack”, or like terms, refers to a relatively sudden deterioration of a chronic condition from a previous state, e.g., a subject's usual state of health, e.g., a stable state. Exacerbations are a common feature of many chronic diseases. An exacerbation typically becomes manifest over a period of up to a few hours, up to a day, up to a week, or up to two weeks. Exacerbations are typically characterized by a marked increase in one or more symptoms of the disorder and/or a marked alteration (indicative of worsening) in one or more physiological parameters relative to the patient's usual state and/or relative to the normal range of the parameter in a healthy individual. An exacerbation of a complement-mediated disorder may result at least in part from and/or be associated with an increased level of complement activation relative to the level of complement activation that is typical for that subject or typical for a healthy individual. In some embodiments a subject who experiences an exacerbation of a complement-mediated disorder may not already be on therapy with a complement inhibitor, e.g., pegcetacoplan. In some embodiments a subject who experiences an exacerbation of a complement-mediated disorder may already be on therapy with a complement inhibitor, e.g., subcutaneously administered pegcetacoplan. The disorder may ordinarily be well controlled by subcutaneously administered pegcetacoplan, but in the event of an exacerbation the subject may benefit from rapid reduction in complement achieved by IV administration of pegcetacoplan as described herein.

In some embodiments a method comprises determining that a subject is experiencing an exacerbation of a complement-mediated disorder (e.g., any of the complement-mediated disorders described herein) and administering a PEGylated compstatin analog, e.g., pegcetacoplan, to the subject intravenously one or more times at a dose of between 500 mg and 2500 mg, e.g., between 1100 mg and 2500 mg, e.g., between 1500 mg and 2500 mg, e.g., about 1500 mg or about 2300 mg. In some embodiments a dose of about 2160 mg may be administered. In some embodiments a dose of between 990 mg and 1215 mg e.g., 1080 mg, may be administered. In some embodiments a dose of between 1215 mg and 2000 mg, e.g., 1500 mg, may be administered. In some embodiments a dose of between 2000 mg and 2500 mg, e.g., 2160 mg or 2300 mg, may be administered. In some embodiments a dose may be selected based at least in part on the duration of complement inhibition desired. For example, in some embodiments a dose of 1500 mg, may be selected, e.g., to suppress alternative pathway complement activation almost completely or completely (e.g., to undetectable levels) for about 24-36 hours. In some embodiments a dose of about 2300 mg may be selected, e.g., to suppress alternative pathway complement activation almost completely or completely (e.g., to undetectable levels) for about 72-96 hours. In some embodiments, e.g., if a longer duration of complement inhibition is desired for treatment of an exacerbation, a subject may be treated with 2 or more IV doses, e.g., 2, 3, 4, or 5 doses, wherein the doses are administered on consecutive days or every other day or every three days. A subject who receives IV administration of a PEGylated compstatin analog, e.g., IV pegcetacoplan, for treatment of an exacerbation may receive subsequent treatment with the intravenously administered PEGylated compstatin analog, e.g., IV pegcetacoplan for subsequent exacerbations.

In some embodiments, a compstatin analog, e.g., a PEGylated compstatin analog, e.g., pegcetacoplan, is administered to a subject using an intensive dosing regimen prior to an event potentially associated with complement activation (e.g., surgery, vaccination). In some embodiments, prior to such an event (e.g., surgery or vaccination), the subject is treated with an intensive dosing regimen comprising intravenous administration of between about 500 mg and about 2500 mg, e.g., between about 540 mg and about 2160 mg, e.g., between about 1100 mg and about 2500 mg, e.g., between about 1500 mg and about 2500 mg, e.g., about 1500 mg or about 2300 mg of pegcetacoplan. In some embodiments, the intensive dosing regimen comprises intravenous administration of about 1080 mg pegcetacoplan. In some embodiments, the intensive dosing regimen comprises intravenous administration of about 2160 mg pegcetacoplan. In some embodiments, pegcetacoplan is intravenously administered to the subject about 1 day, 2 days, 3 days, 4 days, 5 days, 6 days, or 7 days or more prior to the event potentially associated with complement activation (e.g., surgery, vaccination). In some embodiments, pegcetacoplan is intravenously administered to the subject about 1 day, 2 days, 3 days, 4 days, 5 days, 6 days, or 7 days or less prior to the event potentially associated with complement activation (e.g., surgery, vaccination). In some embodiments, the intensive dosing regimen comprises subcutaneous administration of between about 500 mg and about 2500 mg, e.g., between about 540 mg and about 2160 mg, e.g., between about 1100 mg and about 2500 mg, e.g., between about 1500 mg and about 2500 mg, e.g., about 1500 mg or about 2300 mg of pegcetacoplan every day for 3 consecutive days. In some embodiments, the intensive dosing regimen comprises subcutaneous administration of about 1080 mg pegcetacoplan every day for 3 consecutive days. In some embodiments, the intensive dosing regimen comprises subcutaneous administration of about 2160 mg pegcetacoplan every day for 3 consecutive days. In some embodiments, pegcetacoplan is subcutaneously administered to the subject every day for 3 consecutive days, and the third consecutive day is about 1 day, 2 days, 3 days, 4 days, 5 days, 6 days, or 7 days or more prior to the event potentially associated with complement activation (e.g., surgery, vaccination). In some embodiments, pegcetacoplan is subcutaneously administered to the subject every day for 3 consecutive days, and the third consecutive day is about 1 day, 2 days, 3 days, 4 days, 5 days, 6 days, or 7 days or less prior to the event potentially associated with complement activation (e.g., surgery, vaccination).

In some embodiments, a subject suffering from a complement-mediated disorder exhibits acute hemolysis while already on therapy, e.g., therapy with a complement inhibitor, that ordinarily controls hemolysis in the subject. In some embodiments a method comprises determining that a subject with a complement-mediated disorder is exhibiting acute hemolysis (i.e., identifying the subject as exhibiting acute hemolysis) and administering pegcetacoplan to the subject using an intensive dosing regimen. In some embodiments, the subject is treated with an intensive dosing regimen comprising intravenously administering between about 500 mg and about 2500 mg, e.g., between about 540 mg and about 2160 mg, e.g., between about 1100 mg and about 2500 mg, e.g., between about 1500 mg and about 2500 mg, e.g., about 1500 mg or about 2300 mg of pegcetacoplan to the subject. In some embodiments, the intensive dosing regimen comprises intravenously administering about 1080 mg pegcetacoplan. In some embodiments, the intensive dosing regimen comprises intravenously administering about 2160 mg pegcetacoplan. In some embodiments, the intensive dosing regimen comprises subcutaneously administering between about 500 mg and about 2500 mg, e.g., between about 540 mg and about 2160 mg, e.g., between about 1100 mg and about 2500 mg, e.g., between about 1500 mg and about 2500 mg, e.g., about 1500 mg or about 2300 mg of pegcetacoplan to the subject every day for 3 consecutive days. In some embodiments, the intensive dosing regimen comprises subcutaneously administering about 1080 mg pegcetacoplan every day for 3 consecutive days. In some embodiments, the intensive dosing regimen comprises subcutaneously administering about 2160 mg pegcetacoplan every day for 3 consecutive days. In some embodiments the subject is already being treated with therapy, e.g., therapy with a complement inhibitor, prior to the event of acute hemolysis and continues with such therapy following administration of the intensive dosing regimen.

In some embodiments the complement-mediated disorder is PNH, and the subject exhibits acute hemolysis while already on therapy with a complement inhibitor, e.g., subcutaneous pegcetacoplan, that ordinarily controls hemolysis in the subject. In some embodiments a method comprises determining that a subject with PNH is exhibiting acute hemolysis (i.e., identifying the subject as exhibiting acute hemolysis) and treating the subject with an intensive dosing regimen of pegcetacoplan. In some embodiments, the intensive dosing regimen comprises intravenously administering between about 500 mg and about 2500 mg, e.g., between about 540 mg and about 2160 mg, e.g., between about 1100 mg and about 2500 mg, e.g., between about 1500 mg and about 2500 mg, e.g., about 1500 mg or about 2300 mg of pegcetacoplan to the subject. In some embodiments, the intensive dosing regimen comprises intravenously administering about 1080 mg pegcetacoplan. In some embodiments, the intensive dosing regimen comprises intravenously administering about 2160 mg pegcetacoplan. In some embodiments, the intensive dosing regimen comprises subcutaneously administering between about 500 mg and about 2500 mg, e.g., between about 540 mg and about 2160 mg, e.g., between about 1100 mg and about 2500 mg, e.g., between about 1500 mg and about 2500 mg, e.g., about 1500 mg or about 2300 mg of pegcetacoplan to the subject every day for 3 consecutive days. In some embodiments, the intensive dosing regimen comprises subcutaneously administering about 1080 mg pegcetacoplan every day for 3 consecutive days. In some embodiments, the intensive dosing regimen comprises subcutaneously administering about 2160 mg pegcetacoplan every day for 3 consecutive days. In some embodiments the subject is already being treated with SC pegcetacoplan at a dose of 1080 mg administered twice weekly prior to the event of acute hemolysis and continues with such therapy following administration of the intensive dosing regimen. In some embodiments the subject is already being treated with SC pegcetacoplan at a dose of 1080 mg administered twice weekly prior to the event of acute hemolysis and is treated with pegcetacoplan every three days following administration of the intensive dosing regimen. In some embodiments the subject is already being treated with SC pegcetacoplan at a dose of 1080 mg administered twice weekly prior to the event of acute hemolysis and is treated with pegcetacoplan three times per week (i.e., thrice weekly) following administration of the intensive dosing regimen. In some embodiments the subject is already being treated with SC pegcetacoplan at a dose of 1080 mg administered every three days prior to the event of acute hemolysis and continues with such therapy following administration of the intensive dosing regimen. In some embodiments the subject is already being treated with SC pegcetacoplan at a dose of 1080 mg administered every three days prior to the event of acute hemolysis and is treated with pegcetacoplan three times per week (i.e., thrice weekly) following administration of the intensive dosing regimen. In some embodiments the subject is already being treated with SC pegcetacoplan at a dose of 1080 mg administered three times per week (i.e., thrice weekly) prior to the event of acute hemolysis and continues with such therapy following administration of the intensive dosing regimen, optionally with more frequent SC administration than prior to the event. In some embodiments, the last dose of an intensive dosing regimen is the first dose of a resumed maintenance dose. For example, in some embodiments, a subject is treated on day 1 with an intensive dosing regimen comprising intravenous administration of pegcetacoplan, and day 1 is the first day of a subsequent dosing regimen of weekly, twice weekly, every three days, thrice weekly, etc. In some embodiments, a subject is treated starting on day 1 with an intensive dosing regimen comprising subcutaneous administration of pegcetacoplan for three consecutive days (e.g., on day 1, day 2, and day 3), and day 3 is the first day of a subsequence dosing regimen of weekly, twice weekly, every three days, thrice weekly, etc. In some embodiments, a subject may be determined to be experiencing (e.g., identified as exhibiting) hemolysis (e.g., acute hemolysis) if the subject exhibits a measured LDH level that is at least two times the upper limit of normal (ULN), i.e, at least 2×ULN. In some embodiments, the upper limit of normal is about 225 U/L, e.g., in some embodiments, is 225 U/L. In some embodiments, a subject may be identified as exhibiting hemolysis (e.g., acute hemolysis) if the subject additionally exhibits at least one additional sign or symptom of hemolysis (e.g., decrease in hemoglobin (e.g., decrease of at least 1 g/dL or at least 2 g/dL, or decrease Hb below 10 g/dL), hemoglobinuria, or increased fatigue (e.g., a decrease of at least 3 points on FACIT, where higher values indicate less fatigue)). In some embodiments, a subject may be identified as exhibiting hemolysis (e.g., acute hemolysis) if such subject exhibits at least one new or worsening symptom or sign of hemolysis (e.g., fatigue, hemoglobinuria, abdominal pain, dysphagia, dyspnea, anemia (e.g., hemoglobin <10 grams (g)/deciliter (dL)), major adverse vascular event (including thrombosis), or erectile dysfunction) in the presence of elevated LDH >2 times the upper limit of normal (ULN). In some embodiments, a subject may be identified as exhibiting hemolysis (e.g., acute hemolysis) if such subject exhibits an LDH at least 2×ULN after having an LDH below a predetermined level, e.g., below 1.5×ULN, for a period of time, e.g., at least 4 weeks, at least 8 weeks, at least 12 weeks. A subject so identified may be treated with IV pegcetacoplan as described herein, which treatment may be followed by treatment with SC pegcetacoplan. In some embodiments treatment with subcutaneously administered pegcetacoplan may continue for one or more years, e.g., indefinitely, for treatment of PNH. In some embodiments, a subject exhibits a decrease in LDH level following treatment with an intensive dosing regimen described herein. In some embodiments, a subject exhibits a decrease in LDH level 1 day, 2 days, 3 days, 4 days, 5 days, 6 days, 7 days, 8 days, 10 days, 11 days, 12 days, 13 days, 14 days, and/or 15 days following completion of an intensive dosing regimen. In some embodiments, a subject exhibits a decrease in LDH level of about 10%, about 20%, about 30%, about 40%, about 50%, about 60% at about 2 days following completion of an intensive dosing regimen. In some embodiments, a subject exhibits a decrease in LDH level of about 10%, about 20%, about 30%, about 40%, about 50%, about 60%, about 70%, about 80%, about 90% at about 8 days following completion of an intensive dosing regimen. In some embodiments, a subject exhibits a decrease in LDH level of about 10%, about 20%, about 30%, about 40%, about 50%, about 60%, about 70%, about 80%, about 90% at about 15 days following completion of an intensive doing regimen. In some embodiments, a subject exhibits a decrease in LDH level following SC administration of pegcetacoplan every day for 3 consecutive days. In some embodiments, a subject exhibits a decrease in LDH 1 day, 2 days, 3 days, 4 days, 5 days, 6 days, 7 days, 8 days, 10 days, 11 days, 12 days, 13 days, 14 days, and/or 15 days following SC administration of pegcetacoplan every day for 3 consecutive days. In some embodiments, a subject exhibits a decrease of about 10%, about 20%, about 30%, about 40%, about 50%, about 60% at about 2 days following SC administration of pegcetacoplan every day for 3 consecutive days. In some embodiments, a subject exhibits a decrease of about 10%, about 20%, about 30%, about 40%, about 50%, about 60%, about 70%, about 80%, about 90% at about 8 days following SC administration of pegcetacoplan every day for 3 consecutive days. In some embodiments, a subject exhibits a decrease of about 10%, about 20%, about 30%, about 40%, about 50%, about 60%, about 70%, about 80%, about 90% at about 15 days following SC administration of pegcetacoplan every day for 3 consecutive days. In some embodiments, a subject exhibits a decrease in LDH following IV administration of pegcetacoplan. In some embodiments, a subject exhibits a decrease in LDH 1 day, 2 days, 3 days, 4 days, 5 days, 6 days, 7 days, 8 days, 10 days, 11 days, 12 days, 13 days, 14 days, and/or 15 days following IV administration of pegcetacoplan. In some embodiments, a subject exhibits a decrease of about 10%, about 20%, about 30%, about 40%, about 50%, about 60% at about 2 days following IV administration of pegcetacoplan. In some embodiments, a subject exhibits a decrease of about 10%, about 20%, about 30%, about 40%, about 50%, about 60%, about 70%, about 80%, about 90% at about 8 days following IV administration of pegcetacoplan. In some embodiments, a subject exhibits a decrease of about 10%, about 20%, about 30%, about 40%, about 50%, about 60%, about 70%, about 80%, about 90% at about 15 days following IV administration of pegcetacoplan.

In some embodiments, a subject suffering from a complement-mediated disorder who is treated with a PEGylated compstatin analog intravenously, e.g., IV pegcetacoplan, as described herein is not already being treated with SC pegcetacoplan, and the subject may thereafter begin treatment with SC pegecetacoplan, with the first dose of SC pegcetacoplan being administered on the same days as IV administration. In some embodiments, if the subject's is not already being treated with SC pegcetacoplan the subject may begin treatment with SC pegecetacoplan with the first dose of SC pegcetacoplan being administered on the day after the IV administration. In some embodiments, if the subject's is not already being treated with SC pegcetacoplan the subject may begin treatment with SC pegecetacoplan and the first dose of SC pegcetacoplan may be administered with a gap of one day following the IV dose.

In some embodiments, if a subject who is treated with a PEGylated compstatin analog, e.g., pegcetacoplan by IV administration, as described herein, is already being treated with SC pegcetacoplan (e.g., at a dose of 1080 mg administered twice weekly, every three days, thrice weekly, or weekly) and the subject's next dose of SC pegcetacoplan would normally be administered on the same day as administration of an IV dose, the subject may receive the SC dose according to his or her normal schedule (i.e., on the same day as the IV dose). In some embodiments, if the subject's is already being treated with SC pegcetacoplan and the subject's next dose of SC pegcetacoplan would normally be administered on the same day as administration of an IV dose, the next dose of SC pegecetacoplan may instead be administered on the following day. In some embodiments, if the subject's is already being treated with SC pegcetacoplan and the subject's next dose of SC pegcetacoplan would normally be administered on the same day as administration of an IV dose, the next dose of SC pegecetacoplan may instead be administered with a gap of one day following the IV dose. In some embodiments, if the subject's is already being treated with SC pegcetacoplan and the subject's next dose of SC pegcetacoplan would normally be administered the day after administration of an IV dose, the next dose of SC pegecetacoplan may be delayed by one day. In some embodiments treatment with subcutaneously administered pegcetacoplan may continue for one or more years, e.g., indefinitely, for treatment of a chronic complement-mediated disorder.

In some embodiments a subject who experiences an acute event that triggers complement activation, such as those noted below, e.g., a stroke, myocardial infarction, surgical procedure, trauma, or administration of an agent such as gene therapy vector or other exposure to a foreign substance that triggers complement activation, may be treated with a PEGylated compstatin analog intravenously, e.g., IV pegcetacoplan, as described herein, and such treatment is not followed by further administration of a complement inhibitor, e.g., pegcetacoplan, unless the subject received a new diagnosis of a complement-mediated disorder or experiences another triggering event for complement activation. In some embodiments a subject who experiences an acute event that triggers complement activation, such as a stroke, myocardial infarction, surgical procedure, trauma, or administration of an agent such as gene therapy vector or other exposure to a foreign substance that triggers complement activation may be treated with IV pegcetacoplan as described herein, and such treatment is followed by further administration of a complement inhibitor, e.g., pegcetacoplan (e.g., administered subcutaneously), for a limited period of time, e.g., up to 1, 2, 3, or 4 weeks, e.g., not more than a month.

### IV. Pharmaceutical Compositions

Complement inhibitors, e.g., PEGylated compstatin analogs, described herein can be incorporated into pharmaceutical compositions. Such pharmaceutical compositions are useful for, among other things, administration and delivery to a subject in vivo or ex vivo. In some embodiments, pharmaceutical compositions also contain a pharmaceutically acceptable carrier or excipient. Such excipients include any pharmaceutical agent, e.g., a pharmaceutical agent that does not itself induce an immune response harmful to the individual receiving the composition, and which may be administered without undue toxicity. As used herein the terms “pharmaceutically acceptable” and “physiologically acceptable” mean a biologically acceptable formulation, gaseous, liquid or solid, or mixture thereof, which is suitable for one or more routes of administration, in vivo delivery or contact. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

Pharmaceutical compositions may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In some embodiments, a pharmaceutical composition may be a lyophilized powder.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration can comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, mannitol, or dextran. Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility to allow for the preparation of highly concentrated solutions. In some embodiments, pharmaceutical compositions include sodium acetate (e.g., about 5 mM to about 30 mM, e.g., about 10 mM), NaCl (e.g., about 0.5% to about 2%, e.g., about 0.9%) and water, and have a pH of about 4 to about 7, e.g., about 5. In some embodiments, prior to IV administration, such pharmaceutical compositions are diluted in isotonic saline.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Pharmaceutical compositions and delivery systems appropriate for the compositions, methods and uses of the disclosure are known in the art (see, e.g., Remington: The Science and Practice of Pharmacy. 21st Edition. Philadelphia, PA. Lippincott Williams & Wilkins, 2005).

In some aspects, a unit dose of a compstatin analog, e.g., a PEGylated compstatin analogs (e.g. pegcetacoplan), may contain any of the amounts described herein. In some embodiments the unit dose is more than 540 mg, e.g, at least 541 mg, e.g., at least 545 mg. In some embodiments the unit dose is more than 1080 mg, e.g, at least 1081 mg, e.g., at least 1085 mg. In some embodiments the unit dose is more than 2160 mg, e.g, at least 2161 mg, e.g., at least 2165 mg. In some embodiments the unit dose is less than 540 mg, e.g, at most 539 mg, e.g., at most 535 mg. In some embodiments the unit dose is less than 1080 mg, e.g, at most 1079 mg, e.g., at most 1075 mg. In some embodiments the unit dose is less than 2160 mg, e.g, at most 2159 mg, e.g., at most 2155 mg. In some embodiments the unit dose is between about 540 mg and about 2500 mg, about 545 mg and about 1690 mg, about 630 mg and about 930 mg, about 795 mg and about 885 mg, about 585 mg and about 2510 mg, about 900 mg and about 1395 mg, about 990 mg and about 1215 mg, about 1215 mg and about 1395 mg, about 1080 mg and about 1500 mg, between about 1500 mg and about 2160 mg between about 2160 mg and about 2520 mg, about 1500 mg and about 2500 mg, or about 1080 mg and about 2160 mg. In some embodiments the unit dose is about 540 mg. In some embodiments the unit dose is about 1080 mg. In some embodiments the unit dose is about 2160 mg. In some embodiments the unit dose is about 1500 mg. In some embodiments the unit dose is about 2300 mg. The unit dose may contain an amount within any of the ranges described herein, or any particular value within said range.

In some embodiments, described herein is a container, cartridge, syringe, or drug delivery device comprising such a unit dose. Those skilled in the art, reading the present disclosure, will appreciate that, in accordance with standard practice in the field, a container containing a particular volume, as described herein may include an additional volume sufficient to permit the designated particular volume (e.g., unit dose) to be withdrawn from the container for administration.

PEGylated compstatin analogs described herein can be administered by any suitable route. The route and/or mode of administration can vary depending upon the desired results. Methods and uses of the disclosure include delivery and administration systemically, regionally or locally, or by any route, for example, by injection or infusion. The mode of administration is left to the discretion of the practitioner. Delivery of a pharmaceutical composition in vivo may generally be accomplished via injection using a conventional syringe, although other delivery methods such as convection-enhanced delivery can also be used (see, e.g., U.S. Pat. No. 5,720,720). For example, compositions may be delivered subcutaneously, epidermally, epidurally, intracerebrally, intradermally, intranasally, intrathecally, intraorbitally, intramucosally, intraperitoneally, intravenously, intra-pleurally, subretinally, intraarterially, sublingually, intrahepatically, via the portal vein, and intramuscularly. In some embodiments, administration is via intravenous infusion, e.g., central or peripheral intravenous infusion. A clinician may determine the optimal route for administration.

### V. Diseases, Disorders, and Conditions

Provided technologies are useful for preventing or treating various conditions, disorders or diseases, e.g., a condition, disorder or disease described herein. In some embodiments, the present disclosure provides methods for preventing a condition, disorder or disease, comprising administering to a subject susceptible thereto an effective amount of a compstatin analog described herein (e.g., a PEGylated compstatin analog, e.g., pegcetacoplan) using a dose and/or dosing regimen described herein. In some embodiments, the present disclosure provides methods for treating a condition, disorder or disease, comprising administering to a subject suffering therefrom a therapeutically effective amount of a compstatin analog described herein (e.g., a PEGylated compstatin analog, e.g., pegcetacoplan) using a dose and/or dosing regimen described herein. In some embodiments, the present disclosure provides a method for reducing C3 activation, comprising contacting C3 with a compstatin analog described herein (e.g., a PEGylated compstatin analog, e.g., pegcetacoplan) using a dose and/or dosing regimen described herein. In some embodiments, the present disclosure provides a method for reducing C3 convertase activity, comprising contacting a C3 convertase with a compstatin analog described herein (e.g., a PEGylated compstatin analog, e.g., pegcetacoplan) using a dose and/or dosing regimen described herein. In some embodiments, the present disclosure provides a method for reducing complement activation in a system, comprising administering to the system a compstatin analog described herein (e.g., a PEGylated compstatin analog, e.g., pegcetacoplan) using a dose and/or dosing regimen described herein. In some embodiments, the present disclosure provides a method for reducing C3 activation in a system, comprising administering to the system a compstatin analog described herein (e.g., a PEGylated compstatin analog, e.g., pegcetacoplan) using a dose and/or dosing regimen described herein. In some embodiments, the present disclosure provides a method for reducing C3 convertase activity in a system, comprising administering to the system a compstatin analog described herein (e.g., a PEGylated compstatin analog, e.g., pegcetacoplan) using a dose and/or dosing regimen described herein. In some embodiments, the present disclosure provides a method for reducing generation of C3a in a system, comprising administering to the system a compstatin analog described herein (e.g., a PEGylated compstatin analog, e.g., pegcetacoplan) using a dose and/or dosing regimen described herein. In some embodiments, a system is a plurality of cells, a tissue, or an organ in a subject. In some embodiments, a system is or comprises blood. In some embodiments, a system is an animal. In some embodiments, a system is a human. In some embodiments, a subject is a human.

In some embodiments, a condition, disorder or disease is a complement-mediated condition, disorder or disease. In some embodiments, a condition, disorder or disease is or comprises complement-mediated damage to an organ, tissue, or cells. In some embodiments, a compstatin analog described herein (e.g., a PEGylated compstatin analog, e.g., pegcetacoplan) is administered in combination with another therapeutic agent, e.g., a different complement inhibitor.

### A. Blood-Related Disorders

In some embodiments, a compstatin analog described herein (e.g., a PEGylated compstatin analog, e.g., pegcetacoplan), alone or in combination with one or more additional complement inhibitors described herein, is administered to a subject suffering from, or at risk of, a complement-mediated blood-related disorder, such as paroxysmal nocturnal hemoglobinuria (PNH), atypical hemolytic uremic syndrome (aHUS), autoimmune hemolytic anemia, chronic cold agglutinin disease, HELLP syndrome, and/or warm autoimmune hemolytic anemia using a dose and/or dosing regimen described herein. In some embodiments, a compstatin analog described herein (e.g., a PEGylated compstatin analog, e.g., pegcetacoplan) is administered to a subject suffering from, or at risk of, a complement-mediated disorder that affects the circulatory system using a dose and/or dosing regimen described herein. For example, in some embodiments, the disorder is thrombotic microangiopathy (TMA) or a vasculitis (e.g., IgA vasculitis) or other disorder associated with vessel inflammation, e.g., blood vessel and/or lymph vessel inflammation. In some embodiments, a vasculitis is polyarteritis nodosa, hypocomplementemic urticarial vasculitis, pulmonary vasculitis, Wegener's granulomatosis, giant cell arteritis, Churg-Strauss syndrome, microscopic polyangiitis, pauci-immune vasculitis, Henoch-Schonlein purpura, Takayasu's arteritis, Kawasaki disease, or Behcet's disease. In some embodiments, a disorder is TMA secondary to atypical hemolytic uremic syndrome. In some embodiments, a subject is positive for antineutrophil cytoplasmic antibody (ANCA).

### B. Eye Disorders

In some embodiments, a compstatin analog described herein (e.g., a PEGylated compstatin analog, e.g., pegcetacoplan) is administered to a subject for treatment of a complement-mediated eye disorder, such as macular degeneration (e.g., age-related macular degeneration (AMD) and Stargardt macular dystrophy), diabetic retinopathy, glaucoma, or uveitis (e.g., posterior uveitis or anterior uveitis) using a dose and/or dosing regimen described herein. In some embodiments, a subject suffers from or is at risk of AMD. In some embodiments the AMD is neovascular (wet) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease. For example, in some embodiments, treatment of a subject with GA reduces the rate of retinal cell death. A reduction in the rate of retinal cell death may be evidenced by a reduction in the rate of GA lesion growth in patients treated with a compstatin analog described herein (e.g., a PEGylated compstatin analog, e.g., pegcetacoplan), as compared with control (e.g., patients given a sham administration). In some embodiments, a subject has intermediate AMD. In some embodiments, a subject has early AMD. In some embodiments, a subject with intermediate or early AMD is treated in order to slow or halt progression of the disease. For example, in some embodiments, treatment of a subject with intermediate AMD may slow or prevent progression to an advanced form of AMD (neovascular AMD or GA). In some embodiments, treatment of a subject with early AMD may slow or prevent progression to intermediate AMD. In some embodiments an eye has both GA and neovascular AMD. In some embodiments an eye has GA but not wet AMD.

In some embodiments, a subject has an eye disorder characterized by macular degeneration, choroidal neovascularization (CNV), retinal neovascularization (RNV), ocular inflammation, or any combination of the foregoing. Macular degeneration, CNV, RNV, and/or ocular inflammation may be a defining and/or diagnostic feature of the disorder. Exemplary disorders that are characterized by one or more of these features include, but are not limited to, macular degeneration related conditions, diabetic retinopathy, retinopathy of prematurity, proliferative vitreoretinopathy, uveitis, keratitis, conjunctivitis, and scleritis. In some embodiments, a subject is in need of treatment for ocular inflammation. Ocular inflammation can affect a large number of eye structures such as the conjunctiva (conjunctivitis), cornea (keratitis), episclera, sclera (scleritis), uveal tract, retina, vasculature, and/or optic nerve. Evidence of ocular inflammation can include the presence of inflammation-associated cells such as white blood cells (e.g., neutrophils, macrophages) in the eye, the presence of endogenous inflammatory mediator(s), one or more symptoms such as eye pain, redness, light sensitivity, blurred vision and floaters, etc. Uveitis is a general term that refers to inflammation in the uvea of the eye, e.g., in any of the structures of the uvea, including the iris, ciliary body or choroid. Specific types of uveitis include iritis, iridocyclitis, cyclitis, pars planitis and choroiditis. In some embodiments, the eye disorder is Behcet's disease. In some embodiments, the eye disorder is an eye disorder characterized by optic nerve damage (e.g., optic nerve degeneration), such as glaucoma. Additional eye disorders include, e.g., retinitis pigmentosa, macular edema, Vogt-Koyangi-Harada syndrome, birdshot retino-chorioditis, sympathetic ophthalmia, ocular dicatricial pemphigoid, ocular pemphigus, nonartertic ischemic optic neuropathy, post-operative inflammation, and retinal vein occlusion.

### C. Nervous System Disorders

In some embodiments, a compstatin analog described herein (e.g., a PEGylated compstatin analog, e.g., pegcetacoplan) is used to treat a subject suffering from or at risk of a complement-mediated disorder that affects the nervous system, e.g., the central nervous system (CNS) and/or peripheral nervous system (PNS). In some embodiments, a compstatin analog described herein (e.g., a PEGylated compstatin analog, e.g., pegcetacoplan) is administered to a subject suffering from, or at risk or, a complement-mediated disorder that affects the nervous system (e.g., the central nervous system (CNS) and/or peripheral nervous system (PNS)) using a dose and/or dosing regimen described herein. Examples of such disorders include, e.g., a neurodegenerative disorder such as multiple sclerosis, other demyelinating diseases (e.g., neuromyelits optica or chronic inflammatory demyelinating polyneuropathy (CIDP)), amyotrophic lateral sclerosis, chronic pain, fibromyalgia, stroke, intracerebral hemorrhage, allergic neuritis, diabetic neuropathy, Huntington's disease, schizophrenia, Alzheimer's disease, Parkinson's disease, progressive supranuclear palsy, Lewy body dementia (i.e., dementia with Lewy bodies or Parkinson's disease dementia), frontotemporal dementia, progressive supranuclear palsy, corticobasal syndrome, Pick's disease, mild cognitive impairment, traumatic brain injury, traumatic spinal cord injury, multisystem atrophy, chronic traumatic encephalopathy, Creutzfeldt-Jakob disease, Guillain Barre Syndrome, autoimmune encephalitis, and leptomeningeal metastasis. In some embodiments, a subject suffers from neuropathic pain, e.g., arising from lesions that involve the somatosensory pathways with damage to small fibres in peripheral nerves and/or to the spino-thalamocortical system in the CNS.

In certain embodiments the disorder is stroke and the method comprises administering a PEGylated compstatin analog (e.g., pegcetacoplan) using a dose and/or dosing regimen described herein to a subject who has experienced a stroke shortly after the onset of one or more stroke symptoms, e.g., within about 5 minutes, 10 minutes, 15 minutes, 30 minutes, 1 hour, 2 hours, 3 hours, or 4 hours of onset of one or more stroke symptoms (e.g., sudden onset of weakness, blurred or otherwise impaired vision, slurred or otherwise impaired speech). In certain embodiments the disorder has an acute onset, wherein symptoms appear over a period of minutes or hours (e.g., over a period of 24 hours or less, or over a period of 48 hours or less). In some embodiments, the method comprises administering a PEGylated compstatin analog using a dose and/or dosing regimen described herein to a subject who has experienced a stroke shortly (e.g., within about 5 minutes, 10 minutes, 15 minutes, 30 minutes, 1 hour, 2 hours, 3 hours, or 4 hours) after a diagnosis of stroke.

### D. Kidney Disorders

In some embodiments, a compstatin analog described herein (e.g., a PEGylated compstatin analog, e.g., pegcetacoplan) is used to treat a subject suffering from, or at risk of, a complement-mediated kidney disorder. In some embodiments, a compstatin analog described herein (e.g., a PEGylated compstatin analog, e.g., pegcetacoplan) is administered to a subject suffering from, or at risk or, a complement-mediated kidney disorder using a dose and/or dosing regimen described herein. Such disorders include, e.g., nephritis, e.g., glomerulonephritis, e.g., membranoproliferative glomerulonephritis (MPGN) (e.g., MPGN type I, MPGN type II, or MPGN type III), e.g., immune complex membranoproliferative glomerulonephritis (IC-MPGN). In some embodiments the disorder is IgA nephropathy (IgAN), primary membranous nephropathy, or diabetic nephropathy. In some embodiments, the disorder is polycystic kidney disease (PKD). In some embodiments, the disorder is C3 glomerulopathy. In some embodiments the disorder is characterized by glomerular deposits containing one or more complement activation products, e.g., C3b, in the kidney. In some embodiments treatment as described herein reduces the level of such deposits. In some embodiments a subject suffering from a complement-mediated kidney disorder suffers from proteinuria (an abnormally high level of protein in the urine) and/or an abnormally low glomerular filtration rate (GFR). In some embodiments treatment as described herein results in decreased proteinuria and/or an increased or stabilized GFR.

### E. Respiratory Disorders

In some embodiments, a compstatin analog described herein (e.g., a PEGylated compstatin analog, e.g., pegcetacoplan) is used to treat a subject suffering from or at risk of a complement-mediated respiratory disorder. In some embodiments, a compstatin analog described herein (e.g., a PEGylated compstatin analog, e.g., pegcetacoplan) is administered to a subject suffering from, or at risk or, a complement-mediated respiratory disorder using a dose and/or dosing regimen described herein. In some embodiments, a subject is suffering from or at risk of acute respiratory distress syndrome. In some embodiments, a respiratory disease is, e.g., asthma (e.g., allergic asthma), emphysema, chronic inflammation, chronic obstructive pulmonary disease (COPD), pulmonary fibrosis (e.g., idiopathic pulmonary fibrosis), radiation-induced lung injury, allergic bronchopulmonary aspergillosis, hypersensitivity pneumonitis (also known as allergic alveolitis), eosinophilic pneumonia, interstitial pneumonia, sarcoid, Wegener's granulomatosis, pulmonary embolisms and infarcts, dyspnea, hemoptysis, bronchoconstriction, or bronchiolitis obliterans.

### F. Musculoskeletal Disorders

In some embodiments, a compstatin analog described herein (e.g., a PEGylated compstatin analog, e.g., pegcetacoplan) is used to treat a subject suffering from, or at risk of, a complement-mediated disorder that affects the musculoskeletal system. In some embodiments, a compstatin analog described herein (e.g., a PEGylated compstatin analog, e.g., pegcetacoplan) is administered to a subject suffering from, or at risk or, a complement-mediated disorder that affects the musculoskeletal system using a dose and/or dosing regimen described herein. Examples of such disorders include inflammatory joint conditions (e.g., arthritis such as rheumatoid arthritis or psoriatic arthritis, juvenile chronic arthritis, spondyloarthropathies Reiter's syndrome, gout). In some embodiments, a musculoskeletal system disorder results in symptoms such as pain, stiffness and/or limitation of motion of the affected body part(s). Inflammatory myopathies include dermatomyositis, polymyositis, and various others are disorders of chronic muscle inflammation of unknown etiology that result in muscle weakness. In some embodiments, a complement-mediated musculoskeletal disorder is myasthenia gravis.

### G. Transplantation

In some embodiments, a compstatin analog described herein (e.g., a PEGylated compstatin analog, e.g., pegcetacoplan) is used to protect a graft from complement-mediated damage. A graft can be contacted with a compstatin analog described herein (e.g., a PEGylated compstatin analog, e.g., pegcetacoplan) prior to, during, and/or after being transplanted, in various embodiments of the disclosure. In another embodiment, a compstatin analog described herein (e.g., a PEGylated compstatin analog, e.g., pegcetacoplan) is administered to a donor prior to removal of the graft (e.g., using a dose and/or dosing regimen described herein). In some embodiments, a compstatin analog described herein (e.g., a PEGylated compstatin analog, e.g., pegcetacoplan) is administered to a recipient during and/or after the introduction of the graft (e.g., using a dose and/or dosing regimen described herein). In some embodiments, a compstatin analog described herein (e.g., a PEGylated compstatin analog, e.g., pegcetacoplan) is administered to a recipient prior to the introduction of the graft (e.g., using a dose and/or dosing regimen described herein). In some embodiments, a subject receives a compstatin analog described herein (e.g., a PEGylated compstatin analog, e.g., pegcetacoplan) after receiving the graft (e.g., using a dose and/or dosing regimen described herein).

In some embodiments, a graft is or comprises a solid organ such as a kidney, liver, lung, pancreas, or heart. In some embodiments, a graft is or comprises bone, cartilage, fascia, tendon, ligament, cornea, sclera, pericardium, skin, heart valve, blood vessel, amniotic membrane, or dura mater. In some embodiments, a graft comprises multiple organs such as a heart-lung or pancreas-kidney graft. In some embodiments, a graft comprises less than a complete organ or tissue. For example, a graft may contain a portion of an organ or tissue, e.g., a liver lobe, section of blood vessel, skin flap, or heart valve. In some embodiments, a graft comprises a preparation comprising isolated cells or tissue fragments that have been isolated from their tissue of origin but retain at least some tissue architecture, e.g., pancreatic islets. In some embodiments, a preparation comprises isolated cells that are not attached to each other via connective tissue, e.g., hematopoietic stem cells or progenitor cells derived from peripheral and/or cord blood, or whole blood or any cell-containing blood product such as red blood cells (RBCs) or platelets.

In some embodiments, a graft is a xenograft (i.e., the donor and recipient are of different species), an autograft (i.e., a graft from one part of the body to another part of the body in the same individual), an isograft (i.e., the donor and recipient are genetically identical), or an allograft (i.e., the donor and recipient are genetically non-identical members of the same species).

### H. Ischemia/Reperfusion Injury

Ischemia-reperfusion (I/R) injury is an important cause of tissue damage following trauma and in other conditions associated with temporary disruption of blood flow such as myocardial infarction, stroke, severe infection, vascular disease, aneurysm repair, cardiopulmonary bypass, and transplantation. In the setting of trauma, systemic hypoxemia, hypotension, and local interruption of the blood supply resulting from contusions, compartment syndrome, and vascular injuries cause ischemia that damages metabolically active tissues. Restoration of the blood supply triggers an intense systemic inflammatory reaction. After reperfusion, all three major complement pathways are activated and, acting cooperatively or independently, are involved in I/R related adverse events affecting numerous organ systems.

In some embodiments a compstatin analog described herein (e.g., a PEGylated compstatin analog, e.g., pegcetacoplan) is administered to a subject (e.g. using a dose and/or dosing regimen described herein) who has recently (e.g., within the preceding 2, 4, 8, 12, 24, or 48 hours) experienced trauma, e.g., trauma that puts the subject at risk of I/R injury, e.g., due to systemic hypoxemia, hypotension, and/or local interruption of the blood supply. In some embodiments, a compstatin analog described herein (e.g., a PEGylated compstatin analog, e.g., pegcetacoplan) may be administered intravascularly (e.g., using a dose and/or dosing regimen described herein), optionally into a blood vessel that supplies an injured body part or directly to the body part. In some embodiments, the subject suffers from spinal cord injury, traumatic brain injury, burn, and/or hemorrhagic shock.

In some embodiments, a compstatin analog described herein (e.g., a PEGylated compstatin analog, e.g., pegcetacoplan) is administered to a subject prior to (e.g., within the preceding 1, 2, 4, 8, 12, 24, or 48 hours), during, or after (e.g., within the subsequent 1, 2, 4, 8, 12, 24, or 48 hours) a surgical procedure, e.g., a surgical procedure that is expected to temporarily disrupt blood flow to a tissue, organ, or portion of the body, e.g. using a dose and/or dosing regimen described herein. Examples of such procedures include cardiopulmonary bypass, angioplasty, heart valve repair/replacement, aneurysm repair, or other vascular surgeries. A compstatin analog described herein (e.g., a PEGylated compstatin analog, e.g., pegcetacoplan) may be administered (e.g., using a dose and/or dosing regimen described herein) prior to, after, and/or during an overlapping time period with the surgical procedure.

In some embodiments, a compstatin analog described herein (e.g., a PEGylated compstatin analog, e.g., pegcetacoplan) is administered to a subject who has suffered an MI, thromboembolic stroke, deep vein thrombosis, or pulmonary embolism using a dose and/or dosing regimen described herein. In some embodiments the compstatin analog is administered within 1, 2, 4, 8, 12, 24, or 48 hours after the event. A compstatin analog described herein (e.g., a PEGylated compstatin analog, e.g., pegcetacoplan) may be administered in combination with a thrombolytic agent such as tissue plasminogen activator (tPA) (e.g., alteplase (Activase), reteplase (Retavase), tenecteplase (TNKase)), anistreplase (Eminase), streptokinase (Kabikinase, Streptase), or urokinase (Abbokinase). In some embodiments, a compstatin analog described herein (e.g., a PEGylated compstatin analog, e.g., pegcetacoplan) may be administered prior to, after, and/or during an overlapping time period with the thrombolytic agent.

### I. Other Disorders

In some embodiments, a compstatin analog described herein (e.g., a PEGylated compstatin analog, e.g., pegcetacoplan) is used to treat a subject suffering from, or at risk of, a complement-mediated disorder that affects the integumentary system. In some embodiments, a compstatin analog described herein (e.g., a PEGylated compstatin analog, e.g., pegcetacoplan) is administered to a subject suffering from, or at risk or, a complement-mediated disorder that affects the integumentary system using a dose and/or dosing regimen described herein. Examples of such disorders include, e.g., atopic dermatitis, psoriasis, pemphigoid, pemphigus, systemic lupus erythematosus, dermatomyositis, scleroderma, sclerodermatomyositis, Sjögren syndrome, and chronic urticaria.

In some embodiments, a compstatin analog described herein (e.g., a PEGylated compstatin analog, e.g., pegcetacoplan) is used to treat a subject suffering from, or at risk of, a complement-mediated disorder that affects the gastrointestinal system, e.g., inflammatory bowel disease, e.g., Crohn's disease or ulcerative colitis. In some embodiments, a compstatin analog described herein (e.g., a PEGylated compstatin analog, e.g., pegcetacoplan) is administered to a subject suffering from, or at risk or, a complement-mediated disorder that affects the gastrointestinal system e.g., inflammatory bowel disease, e.g., Crohn's disease or ulcerative colitis, using a dose and/or dosing regimen described herein.

In some embodiments, a compstatin analog described herein (e.g., a PEGylated compstatin analog, e.g., pegcetacoplan) is used to treat a subject suffering from, or at risk of, a complement-mediated inflammatory disorder, such as rhinosinusitis or myocarditis. In some embodiments, a compstatin analog described herein (e.g., a PEGylated compstatin analog, e.g., pegcetacoplan) is administered to a subject suffering from, or at risk or, a complement-mediated inflammatory disorder, such as rhinosinusitis or myocarditis, using a dose and/or dosing regimen described herein.

In some embodiments, a compstatin analog described herein (e.g., a PEGylated compstatin analog, e.g., pegcetacoplan) is used to treat a subject suffering from, or at risk of, thyroiditis (e.g., Hashimoto's thyroiditis, Graves' disease, post-partum thyroiditis), hepatitis (e.g., hepatitis C), pancreatitis, panniculitis, or MYH9-related disorders. In some embodiments, a compstatin analog described herein (e.g., a PEGylated compstatin analog, e.g., pegcetacoplan) is administered to a subject suffering from, or at risk or, thyroiditis (e.g., Hashimoto's thyroiditis, Graves' disease, post-partum thyroiditis), hepatitis (e.g., hepatitis C), pancreatitis, panniculitis, or MYH9-related disorders, using a dose and/or dosing regimen described herein.

In some embodiments, a compstatin analog described herein (e.g., a PEGylated compstatin analog, e.g., pegcetacoplan) is used to treat (e.g. using a dose and/or dosing regimen described herein) interleukin-2 induced toxicity during IL-2 therapy, myocardial infarction, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, liver fibrosis, fibrogenic dust diseases, nasal polyposis, parasitic diseases, Goodpasture's Syndrome, immune complex-associated inflammation, antiphospholipid syndrome, cancer, periodontitis, gingivitis, or obesity.

In some embodiments, a complement-mediated condition, disorder or disease is complement activation secondary to administration of another therapeutic or diagnostic agent. For example, in some embodiments, a complement-mediated condition, disorder or disease is complement activation secondary to gene therapy (e.g., gene therapy with a viral vector such as an adeno-associated virus (AAV), adenovirus, or lentivirus vector) or complement activation secondary to cell therapy). In some embodiments, a subject suffers from TMA secondary to hematopoietic stem cell transplant (HSCT-TMA). In some embodiments, a subject suffers from drug-induced TMA. In some embodiments, administration of a compstatin analog described herein (e.g., a PEGylated compstatin analog, e.g., pegcetacoplan) described herein prior to and/or following administration of another therapeutic agent may increase efficacy and/or safety of said therapeutic agent.

In some embodiments a complement-mediated condition, disorder or disease is complement activation secondary to exposure of blood to a foreign material, such as occurs during hemodialysis or extracorporeal membrane oxygenation (ECMO). In some embodiments a method comprises comprising administering the PEGylated compstatin analog to a subject (i) prior to the start of a dialysis procedure (e.g., about 2 hours, 1 hour, 30 minutes, or 15 minutes prior to the start of dialysis) and/or (ii) during the dialysis procedure, and/or (iii) after the end of the dialysis procedure (e.g., for about 15 minutes, 30 minutes, 1 hour, or 2 hours after the end of the dialysis procedure). In some embodiments a method comprises comprising administering the PEGylated compstatin analog to a subject (i) prior to the start of ECMO (e.g., about 2 hours, 1 hour, 30 minutes, or 15 minutes prior to the start of ECMO) and/or (ii) during ECMO, and/or (iii) after the end of ECMO (e.g., for about 15 minutes, 30 minutes, 1 hour, or 2 hours after the end of the dialysis procedure). In some embodiments a method of inhibiting complement activation in a subject whose blood is exposed to one or more components of a dialysis or ECMO circuit (e.g., tubing, membranes) comprises intravenously administering a PEGylated compstatin analog to a subject as described herein (e.g., using a dose and/or dosing regimen described herein).

### VI. Combination Therapy

In some aspects, methods of the present disclosure involve administering a compstatin analog described herein (e.g., a PEGylated compstatin analog, e.g., pegcetacoplan), alone or in combination with one or more additional therapies. In some embodiments, one or more additional therapies modulate an immune response. In some embodiments, a compstatin analog described herein (e.g., pegcetacoplan) is administered to a subject already receiving therapy with another immunomodulatory therapy. In some embodiments, another immunomodulatory therapy is administered to a subject receiving a compstatin analog described herein (e.g., e.g., a PEGylated compstatin analog, e.g., pegcetacoplan). In some embodiments, both a compstatin analog described herein (e.g., a PEGylated compstatin analog, e.g., pegcetacoplan) and another immunomodulatory therapy are administered to the subject.

Examples of immunomodulatory therapies include a cancer vaccine, an adoptive T cell or antibody therapy, an immune checkpoint blockade or a combination thereof. In some embodiments, an immunomodulatory therapy includes agents such as interleukins such (e.g., IL-2, IL-7, IL-12); cytokines such as granulocyte-macrophage colony-stimulating factor (GM-CSF), interferons; various chemokines such as CXCL13, CCL26, CXCL7; antagonists of immune checkpoint blockades such as anti-CTLA-4, anti-PD-1, anti-PD-L1, anti-LAG3 and anti-B7-H3; synthetic cytosine phosphate-guanosine (CpG), oligodeoxynucleotides, glucans, modulators of regulatory T cells (Tregs) such as cyclophosphamide, or other immune modulating agents. In one embodiment, an immunomodulatory therapy includes an agonist antibody to 4-IBB (CD 137). In some embodiments, an immunomodulatory therapy is macrophage modulator such as Bindarit. In some embodiments, an immunomodulatory therapy is a TNFα inhibitor such as Humira. In some embodiments administration of a compstatin analog described herein (e.g., e.g., a PEGylated compstatin analog, e.g., pegcetacoplan) may allow for administering a reduced dosing regimen of (e.g., involving a smaller amount in an individual dose, reduced frequency of dosing, reduced number of doses, and/or reduced overall exposure to) a second therapy, as compared to administration of such second therapy alone. Without wishing to be bound by theory, in some embodiments a reduced dosing regimen of a second therapy may avoid one or more undesired adverse effects that could otherwise result.

In some embodiments such a reduced dose can be administered in a smaller volume, or using a lower concentration, or using a longer dosing interval, or any combination of the foregoing, as compared to administration of a compstatin analog described herein (e.g., pegcetacoplan) or a second therapy alone.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1—Ascending Dose Study of Exemplary PEGylated Compstatin Analog

### Methods

Healthy human volunteers were divided into 4 cohorts, with each cohort including 4 subjects receiving the PEGylated compstatin analog depicted in FIG. 1, having a PEG of about 40 kD (“PEG”) and each cohort including 1 subject receiving placebo. The PEGylated compstatin analog is referred to as PEG in the figure. The PEGylated compstatin analog (i.e., PEG) was formulated in acetate-buffered saline (10 mM NaOAc in 0.900 NaCl, pH 5.0) and administered by a single IV bolus injection administered over approximately 30 m using a syringe pump, according to the following dosing schedules:

| | | Mode of Administration (IV |
|---|---|---|
| | | solution of 24.0 mg/mL |
| | | PEGylated compstatin |
| Cohort | Dosing Schedule | analog (“PEG”)) |
| 1 | Single IV infusion of 200 mg | Single syringe of 8.3 mL (10 |
| | (“PEG-200 mg”) or placebo | mL per syringe) |
| 2 | Single IV infusion of 600 mg | Single syringe of 25 mL (27 |
| | (“PEG-600 mg”) or placebo | mL per syringe) |
| 3 | Single IV infusion of 1500 | Single syringes of 32.5 mL |
| | mg (“PEG-1500 mg”) or | and 30 mL (34 mL per |
| | placebo | syringe) |
| 4 | Single IV infusion of 2300 | Single syringes of 47.9 mL |
| | mg (“PEG-2300 mg”) or | (50 mL per syringe) |
| | placebo | |

### Time Points for Analysis

Blood samples for pharmacokinetic analyses of PEGylated compstatin analog (“PEG”) concentration and pharmacodynamic analyses of alternative complement pathway hemolytic activity (AH50), total complement hemolytic activity (CH50), C3 and C3a levels were collected at 15, 30, and 60 min, 4, 8, 12, and 24 hrs, and Days 3, 4, 5, 6, 7, 8, 15, 22, 29, and 43 following dosing. Subjects were monitored during a safety period from Day 2 to 8 by physical examination, ECG, hematology, serum chemistry, monitoring for injection site reaction and treatment emergent adverse events (TEAEs). Follow-up safety assessments were performed on Days 15, 22, 29, and 43.

### Analytical Methods

AH50 Method: AH50 was measured by a hemolytic assay based on lysis of rabbit red blood cells (RA) due to activation of complement on the cell's surface. The AH50 (50% Alternative Complement Hemolytic dose) was determined for each component by adding a limiting amount of the test serum or plasma. For this assay, the buffers are created such as to block activity of the classical pathway, which requires calcium. Serial dilutions of the test specimen were mixed with equal volumes of the RA. This functional assay measured the amount of hemoglobin that was released when the target cells were lysed by the action of complement, and from this, the percentage of the cells that had been lysed was calculated. The major peak of the hemoglobin spectrum was read at 415 nm wavelength. For each assay the run was verified with a five-point standard and five points of characterized QC control.

AH50 Calculations: For verifiable continuity between assay runs, the AH50 assay was performed using a human serum standard with known AH50 activity. Serial dilutions of the standard were used to establish its 50% lysis point (the point where the best-fit line formed from the percent lysis (Y) versus the reciprocal of the dilution of serum (X) reaches 50%). If the dilution series produced lysis that spanned the 50% point, the three points closest to the 50% point were used to determine the slope and intercept of the line by linear regression. If the percent lysis was very low and none of the dilutions produced 50% lysis, then the two highest points in the series were used, because the slope changes drastically as the sigmoid curve reaches its lower limit. Each specimen from the study was diluted in the same manner and individual 50% lysis points determined by linear regression.

C3a Split Product Testing: C3a was measured by ELISA (BD Pharmingen optEIA) using microtiter plates precoated with specific monoclonal antibodies against C3a. The standards, controls and test specimens were diluted and placed in duplicate into the wells and incubated to allow binding of the split products to the antibodies in the well. After washing away unbound proteins, a second anti-split products antibody, conjugated to an enzyme (horseradish peroxidase) was allowed to react with the split products bound to the first antibody on the plate. After an appropriate incubation time and washing, a chromogenic substrate for the enzyme was added to the wells, and the intensity of color that developed was determined spectrophotometrically. Three QC specimens were run on every assay.

C3a ELISA Calculations: The optical densities (OD) for each of the wells containing the standards for the assay were entered as the dependent variables (Y) in a linear regression calculation (Microsoft Excel) in which the concentrations of the standards (provided by the kit manufacturer, BD Pharmingen) were the independent variables (X). The slope (m) and intercept (b) obtained from the regression analysis on a log-log were then used to calculate the concentrations of the unknowns (controls and test specimens). The formula for a straight line was used to calculate the unknown values using a log/log solution: LnY=mLnX+b, solved for X: X=exp (LnY−b)/m*dilution.

C3: C3 levels were measured on the BindingSite SPA Plus. The SPA plus measures protein concentration by a turbidimetric immunoassay. The specimen to be tested is mixed with a fixed concentration of excess polyclonal antibodies to the analyte of interest. This leads to the formation of large antigen-antibody immune complexes. The specimen is diluted to reach a point where the antigen concentration is near the equivalent point with the antibody. When light is passed through the suspension, a portion is transmitted and focused onto a photodiode by an optical lens system. The amount of transmitted light is indirectly proportional to the specific protein concentration in the sample. Concentrations are automatically calculated by reference to a calibration curve. The amount of analyte (C3 which is causing agglutination) in the sample can therefore be easily determined. C3 concentrations are expressed as g/L.

C3 Calculations: For turbidimetric assays, the change in the amount of light absorbed (inverse of amount transmitted) is dependent on the amount of agglutination between the analyte and the specific antibodies. Thereby, the amount of analyte (C3 which is causing agglutination) in the sample is determined.

### Results

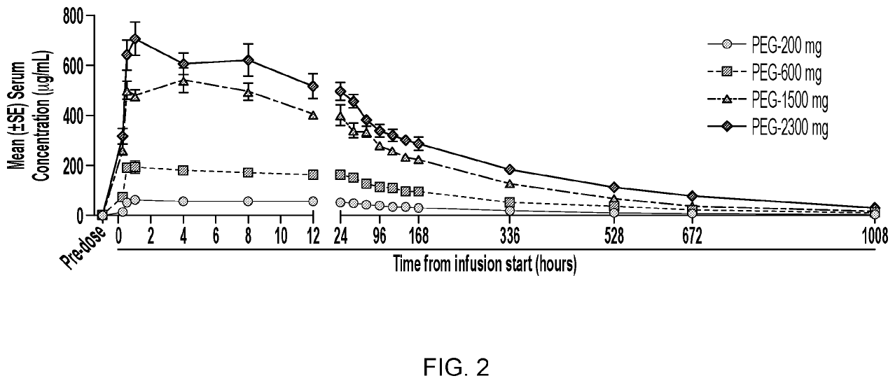

Twenty subjects were enrolled and allocated 4:1 to PEG or placebo per cohort (PEG-200 mg, n=4; PEG-600 mg, n=4; PEG-1500 mg, n=4; PEG-2300 mg, n=4; pooled placebo, n=4). Following a single IV dose, peak concentration (Cₘₐₓ) of PEG was observed at 1 hr post-dose (infusion start) for most cohorts (mean serum concentration: PEG-200 mg, 61 μg/mL; PEG-600 mg, 193 μg/mL; PEG-2300 mg, 708 μg/mL) except PEG-1500 mg (occurred at 4 hrs, 542 μg/mL). PEG concentration at the end of infusion was similar to the observed Cₘₐₓ. PEG concentration declined in a mono-exponential manner, with a terminal elimination half-life ranging from 200 to 222 hrs in Cohorts 1 to 3, increasing to 285 hours in Cohort 4 (FIG. 2). Total body clearance of PEG after IV administration was similar across cohorts.

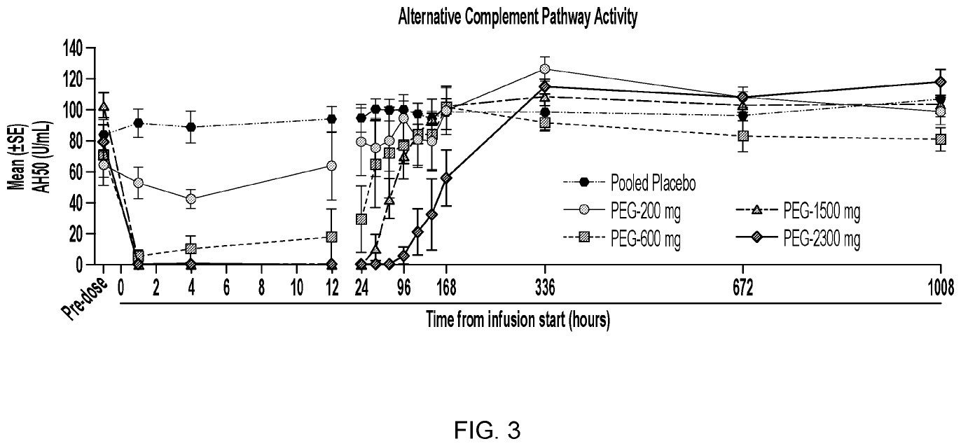

Early, immediate decreases in mean AH50 values were detected within 1 hr in all PEG cohorts, with 1500 and 2300 mg doses decreasing AH50 to undetectable levels from 1 hr post-dosing start (FIG. 3). Decreases in mean AH50 values were maintained for at least 12, 72, 144 and 168 hrs after single doses of 200, 600, 1500 and 2300 mg PEG, respectively. All PEG groups showed a statistically significant larger mean maximum decrease of AH50 compared to placebo (p<0.0001).

Mean CH50 decreased immediately upon dosing in all cohorts (data not shown). Maximum mean decrease from baseline occurred in the PEG-2300 mg group (−71.3 U/mL, −28.23%) at 1 hour post-dosing start and returned to baseline by 24 hours post-dosing start. Decrease in mean CH50 in the PEG-1500 mg group (maximum mean decrease −59.8 U/mL, −23.80%) was retained for at least 48 hours. Only the PEG-1500 mg dose group was statistically significant (p<0.05) with a larger mean maximum decrease in mean CH50 compared to placebo; however, this result may have been skewed by one subject in this group who had a much larger decrease in CH50 at 336 hours post dose.

Mean C3 increased in all PEG groups following a delay, peaking at 168 hours post-dosing start in all the PEG groups except the PEG-600 mg group, which peaked at 120 hours post-dosing start (data not shown). Mean C3 levels remained elevated well beyond Week 4. Maximum mean increase in C3 levels occurred in the PEG-2300 mg group (+1.0923 mg/mL, +90.33%). The mean maximum increase in C3 increased with dose. The PEG-600 mg, PEG-1500 mg and PEG-2300 mg groups all achieved statistically significant larger mean maximum increases of C3 compared to placebo (p<0.05).

All PEG groups had an initial rapid decrease within 1 hr in mean C3a levels, with all dose groups having trough mean C3a levels within 24 hrs of dosing (data not shown). Dose related decreases in mean C3a were not observed, and all doses recorded a max mean decrease of 47% to 57%, with maximum mean C3a decrease from baseline occurring in the PEG-1500 mg group (decrease of 57%) at 4 hours post-dosing start. No changes in C3a levels were seen with placebo, and all PEG groups showed a statistically significant larger mean maximum decrease of C3a compared to placebo (p<0.05).

Of the twenty subjects included in the study, 11 (55.0%) experienced a treatment-related adverse event (TEAE). The most common TEAEs in the PEG groups were headache, (n=6, 37.5%); upper respiratory infections attributed to seasonal viral infection (n=2, 12.5%); diarrhea (n=2, 12.5%). No serious adverse events, deaths, or severe TEAEs occurred. One subject (5.0%) in the PEG-2300 mg cohort experienced a moderate TEAE (infusion-related reaction, dizziness, clamminess, nausea) that led to study discontinuation.

These results suggest that administration of IV PEG in a sodium acetate solution has a favorable safety profile and effectively increases PEG serum concentrations while decreasing complement activity within the first hour post-dose in healthy subjects.

### Example 2—Intensive Dosing Study of Exemplary PEGylated Compstatin Analog

### Methods

Patients who were enrolled in a PNH open-label extension study of pegcetacoplan (including the PEGASUS (NCT03500549), PADDOCK (NCT02588833), and PRINCE (NCT04085601) studies), and who experienced acute hemolysis warranting acute intervention, were offered the opportunity to receive intensive subcutaneous (SC) or intensive intravenous (IV) dosing of the PEGylated compstatin analog depicted in FIG. 1, having a PEG of about 40 kD (“PEG”), at the discretion of the investigator. Eligibility criteria for the intensive dosing regimen included lactate dehydrogenase (LDH) >2× the upper limit of normal (ULN) and one new or worsening sign or symptom of hemolysis (e.g., decreased hemoglobin (Hb), hemoglobinuria, or fatigue).

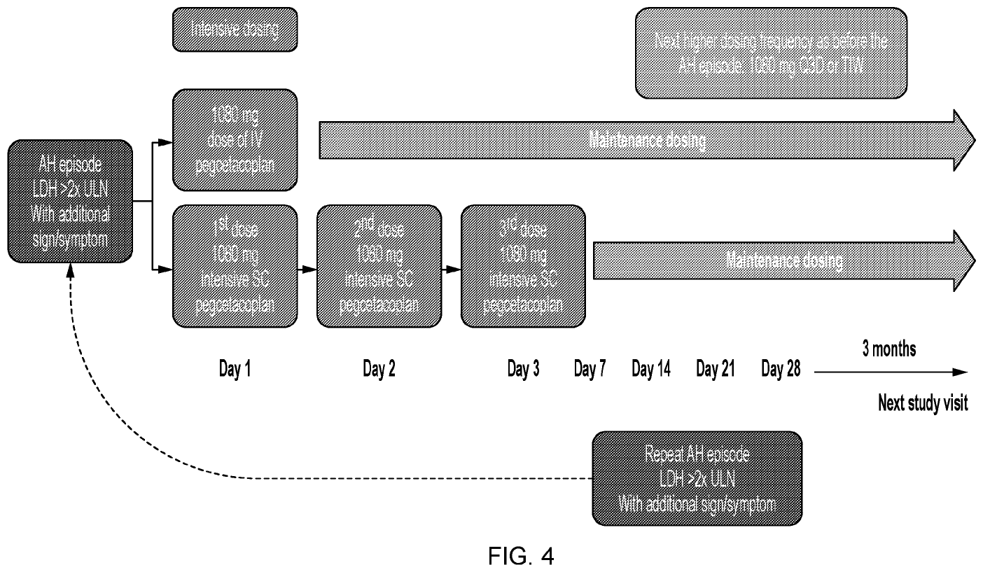

The intensive dosing regimen is shown in FIG. 4. Patients receiving 1080 mg PEG SC twice weekly, and who exhibited acute hemolysis, received a single dose of 1080 mg IV PEG, or 1080 mg SC PEG every 24 hours for 3 doses (intensive PEG treatment), followed by an increased maintenance regimen of 1080 mg SC PEG every 3 days. Patients receiving 1080 mg SC PEG every 3 days or 3 times weekly, and who exhibited acute hemolysis, received intensive PEG treatment followed by maintenance doses of 1080 mg SC PEG 3 times weekly. Patients who experienced additional acute hemolysis events after receiving intensive PEG treatment received additional doses of intensive SC PEG or intensive IV PEG. Further, 4 of 13 patients (3 of 9 intensive SC PEG and 1 of 4 intensive IV PEG treated patients) received at least one red blood cell (RBC) transfusion during the management of the event of acute hemolysis (between day 1-19). Analyzed data relates to the first event if a patient experienced multiple acute hemolysis events.

Levels of Hb (on day 1) and LDH (on day 1, day 2, days 7-12, days 14-19) during the event of acute hemolysis were determined. For measurements on days 7-12 and days 14-19, the first available measurement ≥7 days and ≥14 days after intensive PEG treatment were used, respectively. For each patient, at least 6 days were between the measurements on day 7-12 and day 14-19. Safety was evaluated by the incidence and severity of adverse events.

### Results

In total, 13 of 137 patients who entered the open-label extension study aged 20 to 72 years received intensive PEG treatment. On entry into the open-label extension study, these 13 patients had a mean LDH level of 249 U/L (5 patients >ULN; 11 patients <1.5×ULN) and a mean Hb level of 12.0 g/dL (range: 8-15 g/dL).

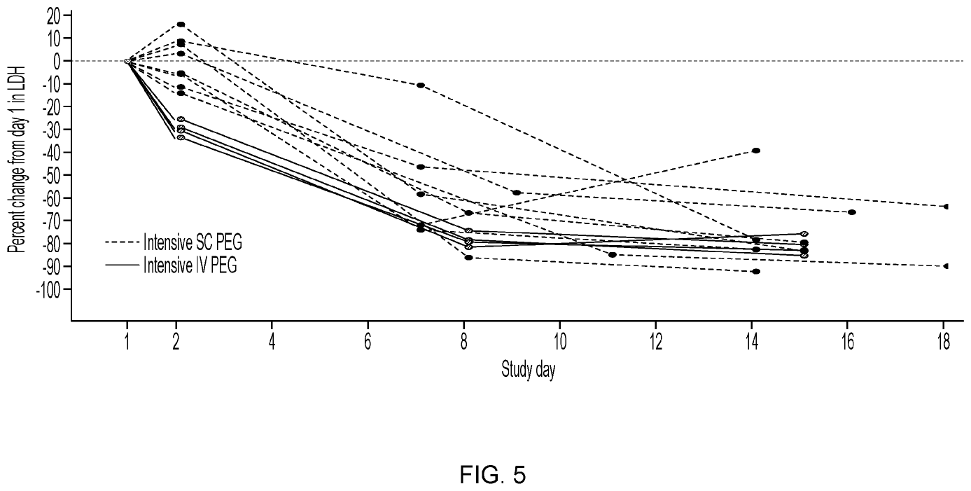

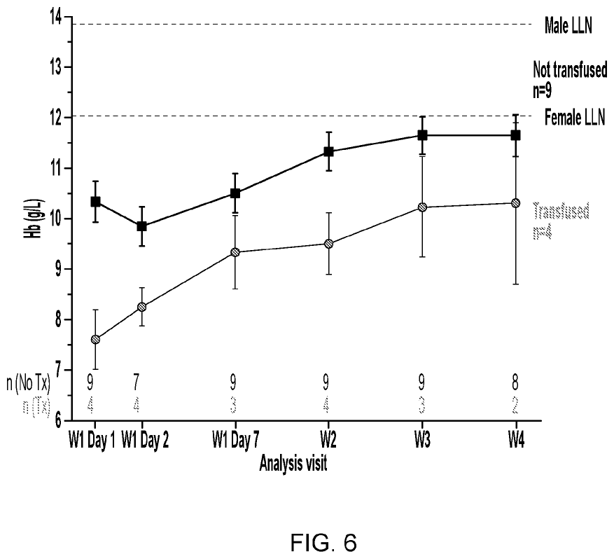

From before intensive PEG treatment to day 1, Hb dropped by <2 g/dL in 6 patients and by >2 g/dL in 7 patients. The largest drop in one patient was 4.9 g/dL (from 11.4 g/dL to 6.5 g/dL). Further, as shown in FIG. 6, mean Hb increased in patients who received at least one RBC transfusion and in patients who did not receive a RBC transfusion. LDH levels were determined by a range of local labs and a central lab; local lab values were standardized to the central lab based on normal ranges from the respective labs. At day 1, 9 patients had LDH <10×ULN and 4 patients had LDH >10×ULN (2 patients treated with intensive SC PEG and 2 patients treated with intensive IV PEG patients). Management of the acute hemolysis event included intensive SC PEG dosing in 9 patients and a single intensive IV PEG dose in 4 patients. As shown in FIG. 5, LDH levels decreased between day 1 and day 2 in 8 of 12 evaluable patients (4 of 8 patients treated with intensive SC PEG and 4 of 4 patients treated with intensive IV PEG) and in all 13 patients at day 7-12. LDH levels further decreased from day 7-12 to day 14-19 in 11 of 13 patients (7 of 9 patients treated with intensive SC PEG and 3 of 4 patients treated with intensive IV PEG).

The incidence and severity of adverse events was similar to that seen in the overall open-label extension study. Nine of 13 patients (69%) experienced treatment-emergent adverse events; 4 of 13 patients (31%) experienced serious adverse events. The majority (76%) of treatment-emergent adverse events were mild. No adverse events of meningitis or thrombosis occurred. Among the 4 patients who experienced serious adverse events, 3 experienced a second event of hemolysis leading to another round of intensive treatment. None of the adverse events led to treatment discontinuation.

These results suggest that intensive SC or intensive IV dosing regimens of PEG can provide effective management of acute hemolysis events and potential rapid control of LDH levels. Further, intensive treatment with PEG displayed a favorable safety and tolerability profile.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of inhibiting complement in a subject, comprising intravenously administering to a subject in need thereof about 100 mg to about 2500 mg of a PEGylated compstatin analog comprising a PEG of about 40 kD, wherein complement is inhibited or reduced (e.g., to a level that is about 100%, 90%, 80%, 70%, 60%, 50%, 40%, or lower, relative to a control level) for about 2 hours to about 336 hours after administration.

2. The method of claim 1, comprising intravenously administering a single dose of the PEGylated compstatin analog.

3. The method of claim 2, wherein the single dose is an infusion.

4. The method of any of the preceding claims, comprising intravenously administering about 200 mg, about 600 mg, about 1500 mg, or about 2300 mg of the PEGylated compstatin analog.

5. The method of claim 3 or 4, comprising administering the infusion at a rate of about 6.5 mg/min to about 80 mg/min.

6. The method of claim 5, comprising administering the infusion at a rate of about 6.5 mg/min, about 20 mg/min, about 50 mg/min, or about 75 mg/min.

7. The method of any one of claims 3-6, comprising administering the infusion over a period of about 15 minutes to about 1 hour.

8. The method of claim 3, comprising intravenously administering about 200 mg, about 600 mg, about 1500 mg, or about 2300 mg of the PEGylated compstatin analog over about 30 minutes.

9. The method of claim 1, comprising administering two or more doses of the PEGylated compstatin analog.

10. The method of any one of claims 1-9, wherein complement inhibition is assessed by measuring level of complement activity in a serum sample of the subject.

11. The method of claim 10, wherein level of complement activity is measured using an alternative pathway assay, a classical pathway assay, or both.

12. The method of any one of claims 1-11, wherein the subject has or is at risk of a complement-mediated disorder.

13. The method of claim 12, wherein the complement-mediated disorder is hemolytic anemia, warm antibody autoimmune hemolytic anemia, cold agglutinin disease, C3 glomerulopathy, Paroxysmal Nocturnal Hemoglobinuria (PNH), myasthenia gravis, glomerulonephritis, Neuromyelitis Optica (NMO), Amyotrophic lateral sclerosis (ALS), polyneuropathy, nephropathy, or vasculitis, optionally further comprising subcutaneously administering the PEGylated compstatin analog to the subject subsequent to the intravenous administration step.

14. A method of treating a subject in need of treatment of a complement-mediated disorder, comprising intravenously administering to a subject in need thereof about 100 mg to about 2500 mg of a PEGylated compstatin analog comprising a PEG of about 40 kD, thereby treating the complement-mediated disorder.

15. The method of claim 14, wherein the complement-mediated disorder is hemolytic anemia, warm antibody autoimmune hemolytic anemia, cold agglutinin disease, C3 glomerulopathy, Paroxysmal Nocturnal Hemoglobinuria (PNH), myasthenia gravis, glomerulonephritis, Neuromyelitis Optica (NMO), Amyotrophic lateral sclerosis (ALS), polyneuropathy, nephropathy, or vasculitis.

16. The method of claim 14 or 15, wherein following the administering step, complement in the subject is inhibited or reduced (e.g., to a level that is about 100%, 90%, 80%, 70%, 60%, 50%, 40%, or lower, relative to a control level) for about 2 hours to about 336 hours after administration.

17. The method of any one of claims 14-16, comprising intravenously administering a single dose of the PEGylated compstatin analog.

18. The method of claim 17, wherein the single dose is an infusion.

19. The method of any one of claims 14-18, comprising intravenously administering about 200 mg, about 600 mg, about 1500 mg, or about 2300 mg of the PEGylated compstatin analog.

20. The method of claim 18 or 19, comprising administering the infusion at a rate of about 6.5 mg/min to about 80 mg/min.

21. The method of claim 20, comprising administering the infusion at a rate of about 6.5 mg/min, about 20 mg/min, about 50 mg/min, or about 75 mg/min.

22. The method of any one of claims 18-21, comprising administering the infusion over a period of about 15 minutes to about 1 hour.

23. The method of any one of claims 14-22, comprising intravenously administering about 200 mg, about 600 mg, about 1500 mg, or about 2300 mg of the PEGylated compstatin analog over about 30 minutes.

24. The method of claim 14, comprising administering two or more doses of the PEGylated compstatin analog.

25. The method of any one of claims 14-24, wherein complement inhibition is assessed by measuring level of complement activity in a serum sample of the subject.

26. The method of claim 25, wherein level of complement activity is measured using an alternative pathway assay, a classical pathway assay, or both.

27. The method of claim any one of claims 14-26, wherein the subject is in need of treatment for an exacerbation of the disorder.

28. A method of treating acute hemolysis in a subject suffering from PNH comprising administering intensive therapy to the subject, wherein the intensive therapy comprises (i) intravenously administering between about 540 mg and about 2160 mg of a PEGylated compstatin analog comprising a PEG of about 40 kD to the subject or (ii) subcutaneously administering between about 540 mg and about 2160 mg of a PEGylated compstatin analog comprising a PEG of about 40 kD to the subject each day for three consecutive days.

29. The method of claim 28, wherein the intensive therapy comprises intravenously administering between about 540 mg and about 2160 mg of a PEGylated compstatin analog comprising a PEG of about 40 kD to the subject.

30. The method of claim 29, wherein the intensive therapy comprises intravenously administering about 1080 mg of a PEGylated compstatin analog comprising a PEG of about 40 kD to the subject.

31. The method of claim 30, wherein the intensive therapy comprises subcutaneously administering between about 540 mg and about 2160 mg of a PEGylated compstatin analog comprising a PEG of about 40 kD to the subject each day for three consecutive days.

32. The method of claim 31, wherein the intensive therapy comprises subcutaneously administering about 1080 mg of a PEGylated compstatin analog comprising a PEG of about 40 kD to the subject each day for three consecutive days.

33. The method of any one of claims 28-32 wherein prior to the acute hemolysis, the subject is being treated with about 1080 mg of a PEGylated compstatin analog comprising a PEG of about 40 kD administered subcutaneously twice weekly and after the intensive therapy the subject receives treatment with about 1080 mg of a PEGylated compstatin analog comprising a PEG of about 40 kD administered subcutaneously every 3 days.

34. The method of any one of claims 28-32 wherein prior to the acute hemolysis, the subject is being treated with about 1080 mg of a PEGylated compstatin analog comprising a PEG of about 40 kD administered subcutaneously every 3 days, and after the intensive therapy the subject receives treatment with about 1080 mg a PEGylated compstatin analog comprising a PEG of about 40 kD administered subcutaneously three times per week.

35. The method of any one of claims 28-32 wherein prior to the acute hemolysis, the subject is being treated with a C5 inhibitor, and after the intensive therapy the subject receives treatment with about 1080 mg of a PEGylated compstatin analog comprising a PEG of about 40 kD administered subcutaneously twice weekly, every 3 days or three times per week.

36. The method of any one of claims 28-32 wherein prior to the acute hemolysis, the subject is being treated with a C5 inhibitor, and after the intensive therapy the subject receives treatment with about 1080 mg of a PEGylated compstatin analog comprising a PEG of about 40 kD administered subcutaneously twice weekly.

37. The method of claim 35 or claim 36 wherein the subject continues treatment with the C5 inhibitor for 4 weeks after receiving the intensive therapy and then discontinues treatment with the C5 inhibitor.

38. The method of any one of claims 28-32 wherein prior to the acute hemolysis, the subject is not being treated with a complement inhibitor, and after the intensive therapy the subject receives treatment with about 1080 mg of a PEGylated compstatin analog comprising a PEG of about 40 kD administered subcutaneously twice weekly, every 3 days or three times per week.

39. The method of claim 38, wherein prior to the acute hemolysis, the subject is not being treated with a complement inhibitor, and after the intensive therapy the subject receives treatment with about 1080 mg of a PEGylated compstatin analog comprising a PEG of about 40 kD administered subcutaneously twice weekly.

40. The method of any one of claims 28-39, wherein prior to the intensive therapy the subject had an LDH level at least 2×ULN.

41. The method of any one of claims 28-40, further comprising determining that the subject is experiencing acute hemolysis by a method comprising detecting an LDH level of at least 2×ULN in a blood sample obtained from the subject prior to administration of the intensive therapy.

42. The method of any one of claims 28-41, further comprising measuring LDH in a blood sample obtained from the subject within 2 weeks after the intensive therapy.

43. The method of any one of claims 1-42, wherein the PEGylated compstatin analog comprises a PEG having at least two compstatin analog moieties attached thereto.

44. The method of claim 43, wherein the PEGylated compstatin analog comprises a linear PEG having a compstatin analog moiety attached to each end.

45. The method of claim 43 or 44, wherein each compstatin analog moiety comprises a cyclic peptide that comprises the amino acid sequence of one of SEQ ID NOs: 3-36, 37, 69, 70, 71, and 72.

46. The method of any one of claims 43-45, wherein the PEGylated compstatin analog comprises one or more PEG moieties attached to one or more compstatin analog moieties, wherein: each compstatin analog moiety comprises a cyclic peptide having an amino acid sequence as set forth in any of SEQ ID NOs:3-36, extended by one or more terminal amino acids at the N-terminus, C-terminus, or both, wherein one or more of the amino acids has a side chain comprising a primary or secondary amine and is separated from the cyclic peptide by a rigid or flexible spacer optionally comprising an oligo(ethylene glycol) moiety; and each PEG is covalently attached via a linking moiety to one or more compstatin analog moieties, and wherein the linking moiety comprises an unsaturated alkyl moiety, a moiety comprising a nonaromatic cyclic ring system, an aromatic moiety, an ether moiety, an amide moiety, an ester moiety, a carbonyl moiety, an imine moiety, a thioether moiety, and/or an amino acid residue.

47. The method of any one of claims 43-46, wherein each compstatin analog moiety comprises a cyclic peptide extended by one or more amino acids at the N-terminus, C-terminus, or both, wherein the one or more amino acids is separated from the cyclic portion of the peptide by a rigid or flexible spacer that comprises 8-amino-3,6-dioxaoctanoic acid (AEEAc) or 11-amino-3,6,9-trioxaundecanoic acid.

48. The method of any one of claims 43-47, wherein the cyclic peptide comprises the amino acid sequence of SEQ ID NO:28, and wherein the spacer comprises AEEAc.

49. The method of any one of claims 1-48, wherein the PEGylated compstatin analog comprises the structure depicted in FIG. 1.


SEQUENCE LISTING (72 sequences)

SEQ ID NO: 1-7 - skipped (7 entries)

SEQ ID NO: 8 - protein, 13 aa, synthetic construct
  SITE 13: note = C-term CONH2 - threonine amide
ICVVQDWGHHRCT

SEQ ID NO: 9 - protein, 13 aa, synthetic construct
  SITE 1: note = N-term Ac - acetyl isoleucine
  SITE 13: note = C-term CONH2 - threonine amide
ICVVQDWGHHRCT

SEQ ID NO: 10 - protein, 13 aa, synthetic construct
  SITE 1: note = N-term Ac - acetyl isoleucine
  SITE 13: note = C-term CONH2 - threonine amide
ICVYQDWGAHRCT

SEQ ID NO: 11 - protein, 13 aa, synthetic construct
  SITE 1: note = N-term Ac - acetyl isoleucine
ICVWQDWGAHRCT

SEQ ID NO: 12 - protein, 13 aa, synthetic construct
  SITE 1: note = N-term Ac - acetyl isoleucine
  SITE 13: note = C-term CONH2 - threonine amide
ICVWQDWGAHRCT

SEQ ID NO: 13 - protein, 13 aa, synthetic construct
  SITE 1: note = N-term Ac - acetyl isoleucine
  SITE 13: note = D-Threonine
ICVWQDWGAHRCT

SEQ ID NO: 14 - protein, 13 aa, synthetic construct
  SITE 1: note = N-term Ac - acetyl isoleucine
  MOD_RES 4: note = 2-Naphthylalanine
  SITE 13: note = C-term CONH2 - threonine amide
ICVXQDWGAHRCT

SEQ ID NO: 15 - protein, 13 aa, synthetic construct
  SITE 1: note = N-term Ac - acetyl isoleucine
  MOD_RES 4: note = 2-Naphthylalanine
ICVXQDWGAHRCT

SEQ ID NO: 16 - protein, 13 aa, synthetic construct
  SITE 1: note = N-term Ac - acetyl isoleucine
  MOD_RES 4: note = 1-Naphthylalanine
ICVXQDWGAHRCT

SEQ ID NO: 17 - protein, 13 aa, synthetic construct
  SITE 1: note = N-term Ac - acetyl isoleucine
  MOD_RES 4: note = 2-Indanyl-Glycine
  SITE 13: note = C-term CONH2 - threonine amide
ICVXQDWGAHRCT

SEQ ID NO: 18 - protein, 13 aa, synthetic construct
  SITE 1: note = N-term Ac - acetyl isoleucine
  MOD_RES 4: note = 2-Indanyl-Glycine
ICVXQDWGAHRCT

SEQ ID NO: 19 - protein, 13 aa, synthetic construct
  SITE 1: note = N-term Ac - acetyl isoleucine
  MOD_RES 4: note = Dihydrotryptophan
ICVXQDWGAHRCT

SEQ ID NO: 20 - protein, 13 aa, synthetic construct
  SITE 1: note = N-term Ac - acetyl isoleucine
  MOD_RES 4: note = Benzoyl-phenylalanine
ICVXQDWGAHRCT

SEQ ID NO: 21 - protein, 13 aa, synthetic construct
  SITE 1: note = N-term Ac - acetyl isoleucine
  MOD_RES 4: note = Benzoyl-phenylalanine
  SITE 13: note = C-term CONH2 - threonine amide
ICVXQDWGAHRCT

SEQ ID NO: 22 - protein, 13 aa, synthetic construct
  SITE 1: note = N-term Ac - acetyl isoleucine
  MOD_RES 4: note = Benzothienyl alanine
ICVXQDWGAHRCT

SEQ ID NO: 23 - protein, 13 aa, synthetic construct
  SITE 1: note = N-term Ac - acetyl isoleucine
  MOD_RES 4: note = Benzothienyl alanine
  SITE 13: note = C-term CONH2 - threonine amide
ICVXQDWGAHRCT

SEQ ID NO: 24 - protein, 13 aa, synthetic construct
  SITE 1: note = N-term Ac - acetyl isoleucine
  MOD_RES 9: note = 2-Aminobutyric acid
  SITE 13: note = C-term CONH2 - threonine amide
ICVWQDWGXHRCT

SEQ ID NO: 25 - protein, 16 aa, synthetic construct
GICVWQDWGAHRCTAN

SEQ ID NO: 26 - protein, 13 aa, synthetic construct
  SITE 1: note = N-term Ac - acetyl isoleucine
  MOD_RES 4: note = 5-fluorotryptophan
  SITE 13: note = C-term CONH2 - threonine amide
ICVWQDWGAHRCT

SEQ ID NO: 27 - protein, 13 aa, synthetic construct
  SITE 1: note = N-term Ac - acetyl isoleucine
  MOD_RES 4: note = 5-methyl-tryptophan
  SITE 13: note = C-term CONH2 - threonine amide
ICVWQDWGAHRCT

SEQ ID NO: 28 - protein, 13 aa, synthetic construct
  SITE 1: note = N-term Ac - acetyl isoleucine
  MOD_RES 4: note = 1-methyl-tryptophan
  SITE 13: note = C-term CONH2 - threonine amide
ICVWQDWGAHRCT

SEQ ID NO: 29 - protein, 13 aa, synthetic construct
  SITE 1: note = N-term Ac - acetyl isoleucine
  MOD_RES 7: note = 5-fluorotryptophan
  SITE 13: note = C-term CONH2 - threonine amide
ICVWQDWGAHRCT

SEQ ID NO: 30 - protein, 13 aa, synthetic construct
  SITE 1: note = N-term Ac - acetyl isoleucine
  MOD_RES 4: note = 5-fluorotryptophan
  MOD_RES 7: note = 5-fluorotryptophan
  SITE 13: note = C-term CONH2 - threonine amide
ICVWQDWGAHRCT

SEQ ID NO: 31 - protein, 13 aa, synthetic construct
  SITE 1: note = N-term Ac - acetyl isoleucine
  MOD_RES 4: note = 5-methyl-tryptophan
  MOD_RES 7: note = 5-fluorotryptophan
  SITE 13: note = C-term CONH2 - threonine amide
ICVWQDWGAHRCT

SEQ ID NO: 32 - protein, 13 aa, synthetic construct
  SITE 1: note = N-term Ac - acetyl isoleucine
  MOD_RES 4: note = 1-methyl-tryptophan
  MOD_RES 7: note = 5-fluorotryptophan
  SITE 13: note = C-term CONH2 - threonine amide
ICVWQDWGAHRCT

SEQ ID NO: 33 - protein, 15 aa, synthetic construct
  MOD_RES 5: note = 6-fluorotryptophan
  MOD_RES 8: note = 6-fluorotryptophan
GICVWQDWGAHRCTN

SEQ ID NO: 34 - protein, 13 aa, synthetic construct
  SITE 1: note = N-term Ac - acetyl isoleucine
  MOD_RES 4: note = 1-formyl-tryptophan
  SITE 13: note = C-term CONH2 - threonine amide
ICVWQDWGAHRCT

SEQ ID NO: 35 - protein, 13 aa, synthetic construct
  SITE 1: note = N-term Ac - acetyl isoleucine
  MOD_RES 4: note = 1-methyoxy-tryptophan
  SITE 13: note = C-term CONH2 - threonine amide
ICVWQDWGAHRCT

SEQ ID NO: 36 - protein, 15 aa, synthetic construct
  MOD_RES 5: note = 5-fluorotryptophan
  MOD_RES 8: note = 5-fluorotryptophan
GICVWQDWGAHRCTN

SEQ ID NO: 37-72 - skipped (36 entries)